(19) United States
(12) Patent Application Publication
(10) Pub. No.: US 2017/0311870 A1
(43) Pub. Date: Nov. 2, 2017

(54) METHOD AND APPARATUS FOR PROCESSING ELECTROENCEPHALOGRAM (EEG) SIGNALS

(71) Applicant: Neurochip Corporation, c/o ZBx Corporation, Toronto (CA)

(72) Inventors: Berj L. BARDAKJIAN, Toronto (CA); Mirna GUIRGIS, Toronto (CA)

(21) Appl. No.: 15/526,547

(22) PCT Filed: Nov. 16, 2015
(86) PCT No.: PCT/CA2015/051191

§ 371 Date: May 12, 2017

Related U.S. Application Data
(60) Provisional application No. 62/080,000, filed on Nov. 14, 2014.

(51) Int. Cl. A61B 5/00; A61B 5/04; A61B 5/0478; A61B 5/048

(52) U.S. Cl. CPC A61B 5/4094; A61B 5/048; A61B 5/04012; A61B 5/0478

(57) ABSTRACT

A method of processing EEC signals received from a plurality electrodes. The method comprises processing the EEC signals to determine a modulation index value for each electrode, determining one or more electrodes that have a modulation index value above a threshold level observed during ictal activity, and using the determined one or more electrodes, to identify one or more possible regions of interest corresponding to seizure zones of a subject's brain.

### FIELD

The subject invention relates to a method and apparatus for processing electroencephalogram (EEG) signals.

### BACKGROUND

The epileptic human brain is one in a chronically hyperexcitable state. This hyperexcitability is realized in the form of neuronal discharges, also known as seizures or ictal events, which can be spontaneous or the result of internal or external stimuli. Excessive communication between various regions of the brain occurs during seizures or ictal events. This communication, which is often long-range, may be facilitated by lower frequencies [43]. Low frequency oscillations (LFOs), which can serve as markers of epileptogenicity, are generally considered rhythms having a frequency oscillation of less than 30 Hz. The delta rhythm (having a frequency oscillation less than 4 Hz) has been found to be a useful marker for lateralizing the epileptogenic focus [41]. Intermittent delta activity has also been found to identify its presence [22]. Specifically, both temporal and occipital intermittent rhythmic delta activity are highly correlated with epilepsy [9].

The presence of delta slow waves, in the absence of spiking activity, was more prevalent in patients with uncontrolled seizures (defined as >2 seizures per month; compared to patients with controlled seizures defined as <2 seizures per year) [23]. Interictal regional delta slowing has also been found to correlate with positive surgical outcomes in patients with temporal lobe epilepsy (TLE) [45]. Although the underlying mechanisms responsible for this slowing are still unknown, it has been suggested that this activity can be used as an EEG marker of TLE networks [45].

Delta activity has also been suggested to be useful for the application of seizure detection using autoregression spectral techniques [34]. Moreover, non-epileptiform activity in the delta and theta (4 to 8 Hz) rhythms has been detected in the same spatial region as epileptiform activity in children with focal epilepsy [50]. In comparison to healthy subjects, patients with partial epilepsy show distinct anatomical theta patterns [14]. Interictal rhythmical midline theta was also found to be significantly more frequent in patients with frontal lobe epilepsy compared to those presenting with TLE and non-epileptic patients [5].

High frequency oscillations (HFOs >80 Hz) have been implicated as main factors in epileptic seizures. Collectively referred to as HFOs along with the gamma rhythm (30 to 80 Hz), the ripples (80 to 200 Hz) and fast ripples (>200 Hz) have been observed in both rodents [35] and humans [10], [26], [40], [49]. Differences between physiological and pathological HFOs have been discussed [18]. The most notable feature separating the two is their spatial origin. Ripples generated from the dentate gyrus can be considered pathological whereas physiological ripples can originate from normal hippocampus or parahippocampal structures. Underlying mechanisms generating these HFOs have been discussed [29]. It has been found that ripples accompanied by continuous/semicontinuous background EEG activity show a higher prevalence in the hippocampus and occipital lobe with no correlation to the seizure onset or lesion sites, suggesting that this is a type of physiological neuronal activity rather than pathological [36]. This rhythm is suggested to be the result of inhibitory field potentials that may be involved in strong coherence of long-range neuronal activity [11], [53]. Fast ripples are generally believed to be pathological. However, some areas of normal neocortex have been found to generate fast ripple oscillations [32], [30], [31]. Thus the frequencies involved in the oscillations are not sole indications of pathological activity. Pathological fast ripples have been suggested to be the result of the strong coherence of abnormally bursting neurons [8]. The difference in the neuronal activity involved and the different spatial origins suggest that the fast ripples are not the harmonics of the ripples [18], as some have stipulated [20]. Studies have also found that the resection of HFO-generating tissue can lead to positive surgical outcome in both adults [29] and children [1], [51]. The increasing potential of HFO-guided resections have been discussed [27].

Ripples have been found to coexist with various background EEG patterns [36]. Surgical resection of the areas generating fast ripples and ripples coexisting with flat background EEG activity were found to significantly correlate with a seizure-free outcome whereas resection of areas generating ripples with a continuously oscillating background EEG pattern showed no positive correlation with post-surgical outcome [33]. Extending beyond coexistence, some have looked at the interactions between different rhythms. Specifically, cross-frequency coupling (CFC) in the form of modulation has been explored as predictive feature of seizures [2].

Clinicians perform a pre-surgical evaluation using both non-invasive and invasive tools [16] to identify the seizure onset zone (SOZ), which is defined as the cortical region generating clinical seizures [42]. Ideally, this region would significantly, if not completely, overlap with the epileptogenic zone (EZ), which is the cortical area that is indispensable for epileptic seizure generation [42]. This region is theoretical in that it cannot be directly measured. The success of the SOZ capturing this area is determined only post operatively by examining the level of improvement of the patient. The highest rates of seizure-free patients at least one year post operatively were 66to100% among those presenting with dual pathologies and the lowest rates were 36to76% among those presenting with localized neocortical epilepsy [44]. This variability in post-surgical outcomes is in large part due to the difficulty and subjectivity involved in defining the SOZ.

It is therefore an object to provide a novel method and apparatus for processing EEG signals.

### SUMMARY

Accordingly, in one aspect there is provided a method of processing electroencephalogram (EEG) signals received from a plurality electrodes, the method comprising processing the EEG signals to determine a modulation index value for each electrode, determining one or more electrodes that have a modulation index value above a threshold level observed during ictal activity, and using the determined one or more electrodes to identify one or more possible regions of interest corresponding to seizure zones of a subject's brain.

In an embodiment, the method comprises using the determined one or more electrodes to identify seizure onset and termination times.

In embodiments, the method comprises cross-frequency coupling the EEG signals. The cross-frequency coupling comprises modulating amplitudes of high-frequency oscillations of the EEG signals by phases of low-frequency oscillations of the EEG signals. The high-frequency oscillations comprise frequencies between 11 Hz and 450 Hz. The low-frequency oscillations comprise frequencies between 0.5 Hz and 10 Hz.

In embodiments, the threshold level is approximately 0.3 times the maximum modulation index.

In embodiments, the method comprises determining one or more electrodes that have the modulation index value above another threshold level for a period of time.

In embodiments, the method comprises using the determined one or more electrodes to calculate cross-electrode modulation indexes. The method further comprises calculating an eigenvector using the calculated cross-electrode modulation indexes, and determining one or more electrodes associated with a component of the eigenvector that is above a threshold.

In embodiments, the method comprises eigenvalue decomposing the calculated cross-electrode modulation index values to determine a number of eigenvalues, calculating a mean of the eigenvalues, and setting the threshold level as three standard errors of mean above the calculated mean of the eigenvalues.

In embodiments, the plurality of electrodes are formed as a grid of electrodes.

According to another aspect there is provided a non-transitory computer-readable medium comprising program code for executing by a processor to process electroencephalogram (EEG) signals received from a plurality of electrodes, the program code comprising program code for processing the EEG signals to determine a modulation index value for each electrode of said grid, program code for determining one or more electrodes that have a modulation index value above a threshold level observed during ictal activity, and program code for using the determined one or more electrodes to identify one or more possible regions of interest corresponding to seizure zones of a subject's brain.

In embodiments, the computer-readable medium further comprises program code for using the determined one or more electrodes to identify seizure onset and termination times.

According to another aspect there is provided an apparatus comprising memory storing executable instructions, and at least one processor communicating with the memory and executing the instructions therein to cause the apparatus at least to receive EEG signals from a plurality of electrodes, process the EEG signals to determine a modulation index value for each electrode, determine one or more electrodes that have a modulation index value above a threshold level observed during ictal activity, and use the determined one or more electrodes to identify one or more possible regions of interest corresponding to seizure zones of a subject's brain.

The subject method and apparatus investigate LFO-HFO cross-frequency coupling (CFC) to identify an area for resection that would potentially result in a seizure-free outcome.

The subject method and apparatus define regions of interest (ROIs). Regions are identified by an automated algorithm [7],[50], encompassing the epileptogenic zone (EZ). The identified regions are compared to the SOZ identified by two independent neurologists. By monitoring the CFC between LFOs and HFOs, ROIs are identified. Specifically, the modulation of the HFOs by the LFOs is examined in the epileptic human brain. Intracranial electroencephalogram (iEEG) recordings obtained from subdural grids implanted in five patients presenting with intractable extratemporal lobe epilepsy (ETLE). The wavelet transform is used to extract rhythms of interest, which are then used to compute the modulation index (MI) as a measure of the strength of phase-amplitude coupling between rhythms of varying frequencies.

### BRIEF DESCRIPTION OF THE DRAWINGS

Embodiments will now be described more fully with reference to the accompanying drawings in which:

FIG. 1 shows a flowchart outlining a method for processing EEG signals obtained from a plurality of electrodes;

FIG. 2 shows a flowchart outlining three methods for identifying potential epileptogenic tissue that were compared, namely, seizure onset zone (SOZ) identification by two independent neurologists, channel selection by visual inspection of MI, and region of interest (ROI) identification by eigenvalue decomposition;

FIG. 3A shows a placement of a 64-electrode grid with 3.0 mm diameter platinum electrodes positioned 10.0 mm apart center-to-center implanted on the cortex of a subject's brain;

FIG. 3B shows the difference between adjacent non-overlapping electrodes of FIG. 3A to provide a local reference, resulting in a 32-channel array;

FIG. 4 shows three referencing schemes that were compared using the same channel (channel 5 on the 32-channel array of FIG. 3B, which is electrode 10 on the 64-electrode grid of FIG. 3A) from the same seizure segment of a Patient A;

FIG. 5 shows the recording from channel 2 of Patient A used as a sample signal to determine if the observed MI was the result of a harmonic effect;

FIGS. 6A, 6B and 6C show four complex mother wavelets compared along with their corresponding MI values computed in the same 10 s window;

FIG. 7A shows a segment of the local field potential (LFP) from channel 2 of Patient A;

FIG. 7B shows a comparison of MI values in three different channels of the same seizure at seizure onset;

FIG. 8 shows MI frames selected at seizure onset, mid-seizure, and seizure termination, the seizure onset and termination frames selected to be at most 10 frames before or after the clinical timestamp, as the MI is computed in 10 s windows;

FIGS. 9A, 9B and 9C show frames selected at seizure onset, mid-seizure and seizure termination, respectively, for Patient D;

FIG. 10 shows cross-channel MI computed for all possible pairings for the three select frames at seizure onset, mid-seizure, and seizure termination for Patient D;

FIGS. 11A and 11B show HFO- and LFO-Centered Cross-Channel MI, respectively;

FIG. 12 shows a matrix of mean significant MI values at seizure onset from Patient D decomposed using EVD;

FIG. 13 shows a graph of global coherence at seizure onset;

FIG. 14 shows the global Coherence (C_{global}) at each MI frame;

FIG. 15 shows multiple ROIs identified for Patient D using all three MI frames;

FIG. 16 shows the channels defining the ROIs identified from all three MI frames for Patient A as well as the overall cumulative summary of these identified channels;

FIG. 17 shows the channels defining the ROIs identified from all three MI frames for Patient B as well as the overall cumulative summary of these identified channels;

FIG. 18 shows the channels defining the ROIs identified from all three MI frames for Patient C as well as the overall cumulative summary of these identified channels;

FIG. 19 shows the channels defining the ROIs identified from all three MI frames for Patient E as well as the overall cumulative summary of these identified channels;

FIG. 20 shows ROIs for Patients A, B, C, and E based on the Seizure Onset Frame; and

FIGS. 21A and 21B show modulation index values calculated based off scalp electrode data recorded during non-seizure and seizure activity based off scalp electrode data.

### DETAILED DESCRIPTION OF THE EMBODIMENTS

In the following, a method, apparatus and computer-readable medium for processing electroencephalogram (EEG) signals obtained from a plurality of electrodes are described.

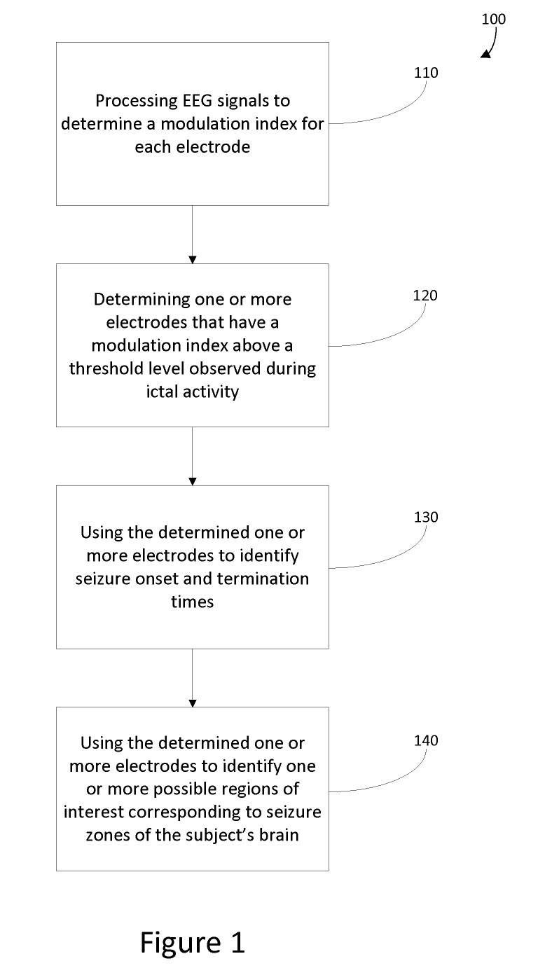

Turning now to FIG. 1, a method for processing EEG signals obtained from a plurality of electrodes is shown and is generally identified by reference numeral 100. The EEG signals are processed to determine a modulation index (MI) value for each electrode (step 110). In this embodiment, each MI value is calculated by determining the distribution of high frequency amplitudes across low frequency phase bins. As will be described in more detail below, the Kullback-Leibler distance is used to compute how skewed the distribution is compared to a uniform distribution. In this embodiment, the MI value is a numerical representation of the level of skewness calculated. As will be appreciated, the MI values range between zero (0) and one (1). One or more electrodes that have a modulation index above a threshold level observed during ictal activity are determined (step 120). In this embodiment, once MI has been calculated for all possible electrode pairings for a set amount of time points, the MI values are used to create matrices. The matrices are eigenvalue decomposed and the resulting eigenvalues are used to identify electrodes above the threshold level. The mean value of the eigenvalues extracted from the decomposition is calculated and as such the threshold is set to be three standard errors of mean above the calculated mean value. The determined electrodes are used to identify seizure onset and termination times (step 130) and to identify one or more possible seizure zones of a subject's brain (step 140).

As will be appreciated, the EEG signals are processed using a suitable computing device. In this embodiment, the computing device is a general purpose computer or other suitable processing device comprising, for example, a processing unit, system memory (volatile and/or non-volatile memory), other non-removable or removable memory (e.g., a hard disk drive, RAM, ROM, EEPROM, CD-ROM, DVD, flash memory, etc.) and a system bus coupling the various computing device components to the processing unit. The memory of the computing device stores program instructions, that when executed, processes EEG signals obtained from a plurality of electrodes as described above. A user may enter input or give commands to the computing device via a mouse, keyboard, touch-screen or other suitable input device. Other input techniques such as voice or gesture-based commands may also be employed.

An example of using method 100 will now be described with reference to iEEG data acquired from a plurality of patients. In particular, iEEG data was collected from five (5) patients: Patient A, Patient B, Patient C, Patient D and Patient E. The iEEG data was processed according to method 100 to determine seizure onset and termination times as well as to identify any potential region(s) of interest (ROI) corresponding to seizure zones. The results of this method were compared to the results determined by two (2) independent neurologists as well as the surgical outcome of the patients who had undergone resective surgery. The method 100 was found to appropriately identify seizure onset and termination times comparable to those identified by the neurologists. The method 100 was able to identify the same region that was ultimately resected in the patients who experienced a positive surgical outcome. The clinical background of each patient is outlined in Table 1.

TABLE 1
Clinical Background of Patient Database

| | | Duration | | Grid | | Surgical |
|---|---|---|---|---|---|---|
| Patient | Age/Sex | (years) | MRI Findings | Placement | Pathology | Outcome |
| A | 36/F | 20 | abnormal intensity | Left: FT | n/a | no |
| | | | lesion, perisylvian | | | resection |
| | | | | | | (eloquent |
| | | | | | | cortex) |
| B | 16/M | 4 | cortical dysplasia | Right: DF | normal | EC IV |
| C | 30/M | 16 | hippocampal atrophy; | Right: NT, F | atypical neuron | EC II |
| | | | dilated perisylvian | | with reactive | |
| | | | | | gliosis | |
| D | 28/M | 16 | non-lesional | Left: FT | Cortical | EC III |
| | | | | | microdysgenesis | |
| E | 56/F | 8 | non-lesional | Left: P | Cortical | EC II |
| | | | | | microdysgenesis | |

N: neocortical;
F: frontal;
T: temporal;
D: dorsolateral;
P: parietal;
EC: Engal Class [19]

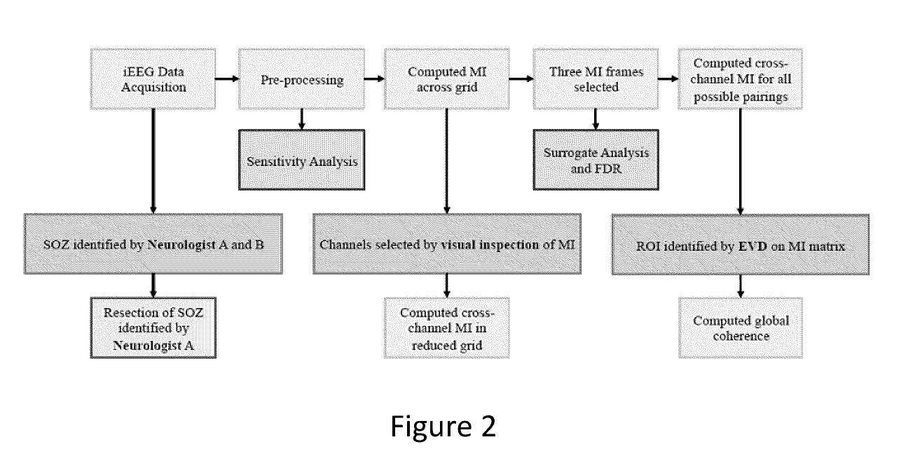

FIG. 2 shows a flowchart outlining the example. As can be seen and as described above, three methods were used for identifying potential epileptogenic tissue. The three methods were seizure onset zone (SOZ) identification by two independent neurologists, channel selection by visual inspection of MI, and region of interest (ROI) identification by eigenvalue decomposition. Surgical resection was performed on the SOZ by Neurologist A. Sensitivity and surrogate analyses, also complimented by applying a false discovery rate (FDR) algorithm, were performed to examine the significance of the modulation observed.

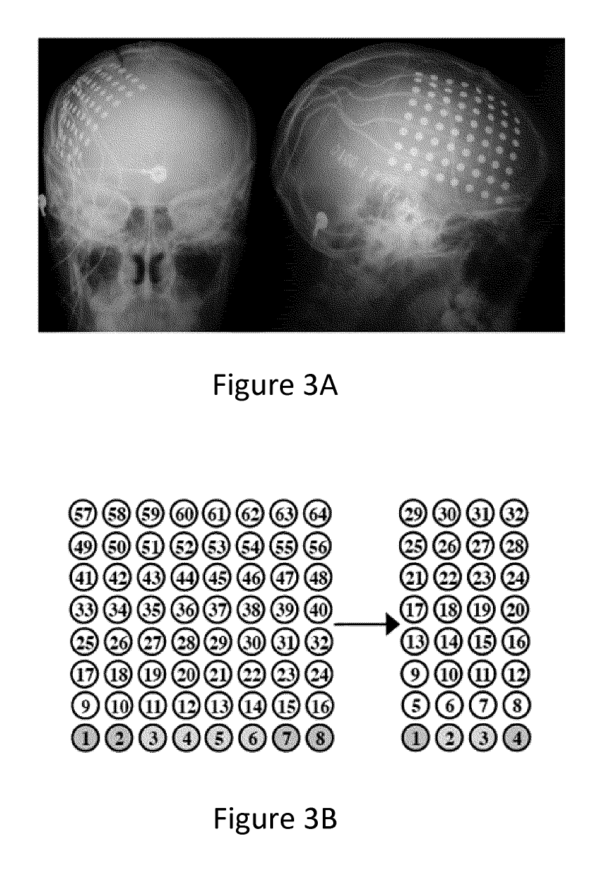

As shown in FIG. 3A, a sixty-four (64) contact (8×8) grid pattern was implanted on the cortex of each patient, made of 3.0 mm diameter platinum electrodes positioned 10.0 mm apart center-to-center. In this example, each electrode was a PMT® Cortac® Cortical Electrode. A local reference was used for each electrode by taking the difference between adjacent non-overlapping electrodes, to minimize ground artifacts that may have been present in the individual electrodes and to reduce the dimensionality of the grid, forming a 32-channel array as shown in FIG. 3B. Recordings were then down-sampled to 1 kHz from 2 kHz after applying an anti-aliasing finite impulse response (FIR) filter. Power line interference was also FIR notch filtered at 50 Hz with all associated harmonics up to 450 Hz. A sensitivity analysis on the parameters selected for these pre-processing measures was performed.

The pre-processing measures taken to prepare the data were investigated by performing a sensitivity analysis on select parameters.

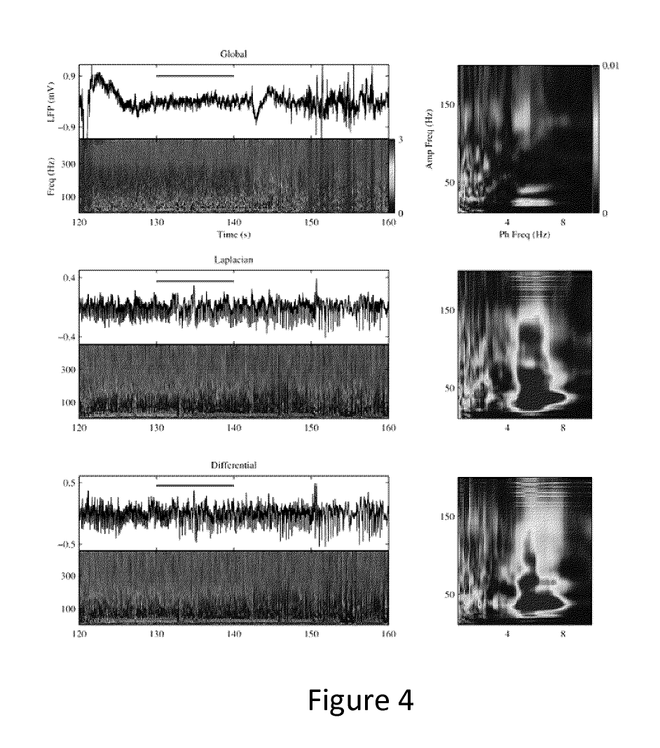

Three references were investigated. The first was a common global reference, which was located either at the forehead or behind the ears and was thus at varying distances from each channel. The second was a local Laplacian reference, which is often used for scalp electrodes [54]. This approach uses the mean potential recorded from the surrounding electrodes as the reference for a center electrode as shown in equation [1]:

$${J}_{i} ( t ) = {V}_{i} ( t ) - \frac{1}{M} {\sum }_{m = 1}^{M} {V}_{i}^{m} ( t ) \qquad [ 1 ]$$

where Vᵢ^{m}(t) is the potential at the m^{th} closest electrode to electrode i. The surrounding surrounding potentials were taken in a symmetrical arrangement. The use of this reference to iEEG was done using potentials from electrodes along the grid lines in the four cardinal directions from a center electrode (i.e., M=4). This symmetry was selected as opposed to using the four electrodes along the diagonals between the four cardinal directions because the distance between the grid line electrodes and the center electrode was smaller than the diagonal distance. The third reference used was the difference between adjacent electrodes (i.e., a differential montage). A sample of the complex wavelet coefficients computed from the time series obtained from each of these three references along with their respective MI values is shown in FIG. 4.

The global reference (top panel of FIG. 4) made use of the reference electrodes placed on the forehead or behind the ears of the subject. The DC shift from the recording was removed from the Laplacian (middle panel of FIG. 4) and differential (bottom panel of FIG. 4) references as well as artifact high amplitude spiking. Horizontal bars over the local field potential (LFP) indicate the 10 s window for which MI was computed and illustrated on the right of each respective panel. The MI resulting from the Laplacian and differential references were comparable whereas the MI resulting from the global reference was significantly weaker and ultimately negligible. The Laplacian reference requires symmetry around each electrode and thus the electrodes on the edges of the grid were discarded. However, differential references provided comparable MI values and allowed for all electrodes to be included in the analysis. Thus, the differential montage was used for analysis. Wavelet power was z-score normalized per frequency (in 1 Hz increments) using the mean and standard deviation of the wavelet power from a 30 s window more than 60 s before seizure onset. This allowed for all frequency activity to be visible on the same scale. MI was computed between the phase of the frequencies indicated on the x-axis and the amplitude of the frequencies indicated on the y-axis. All scales and axes labels are indicated in the top panel of FIG. 4.

The Laplacian and differential references were found to have comparable MI values. However, the requirement of the Laplacian reference for symmetry around each channel resulted in the electrodes on the boundaries of the grid to be discarded. Thus the differential reference was used for the subsequent analysis. FIR filters were used for both lowpass and notch filtering purposes. Filter orders of 50, 500, 5000 and 10000 were compared. A filter order of 5000 was found to have a comparable −55 dB drop at the desired frequencies as the 10000-order filter. Moreover, the filter order was found to not have an effect on the resulting MI. As such, FIR filters with an order of 5000 were used for the analysis.

Downsampling was performed after an anti-aliasing 750 Hz lowpass FIR filter was applied. Two MATLAB® algorithms were compared: resample and decimate. The former interpolates between sample points if necessary, depending on the resampling ratio, while the latter discards sample points to produce a downsampled signal. MI was found to be unaffected by the choice of the downsampling algorithm. As such, resample was used for the analysis.

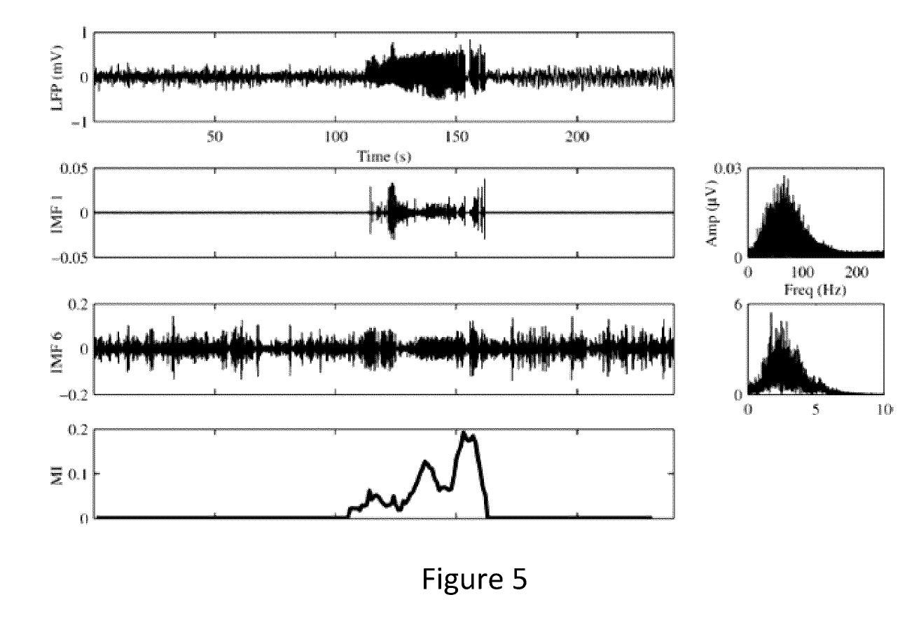

To ensure that the HFO modulation being observed was not the result of harmonics, ensemble empirical mode decomposition (EEMD) was applied to a sample channel to extract the individual rhythms [55], [56]. The resulting intrinsic mode functions (IMFs) are essentially monorhythmic and hence the application of the Hilbert transform to extract the amplitude envelope of the HFO and the instantaneous phase of the LFO was used. The first and sixth IMFs were used for computing MI (FIG. 5).

In FIG. 5, ensemble empirical mode decomposition (EEMD) was applied using a noise standard deviation of 0.1 and N=200 ensembles. The Hilbert transform was applied to the first and sixth intrinsic mode functions (IMF 1 and IMF 6 shown in FIG. 5, respectively) in order to extract the amplitude envelope of the higher rhythm and instantaneous phase of the lower rhythm. Frequency content of each IMF is shown in the fast Fourier transform on each respective row of FIG. 5. As can be seen, the frequency content of IMF 1 peaks around 80 Hz and includes frequencies 150 Hz while IMF 6 peaks around 3 Hz and includes frequencies 5 Hz. The resulting MI is shown in the bottom panel of FIG. 5.

As will be appreciated, the presence of MI after the extraction of a single HFO rhythm (i.e., IMF 1) suggests that the modulation was not due to harmonics. Additionally, to ensure that the observed modulation was not the result of artifactual or physiological spiking activity, a study performing data simulations was examined. The study investigated the effect of spiking activity on MI in eight different scenarios [24]. Observed significant CFC was observed only when HFOs were synchronized to a slower rhythm. Random spiking activity in the presence of HFOs and synchronized spiking activity in the absence of HFOs did not result in significant CFC.

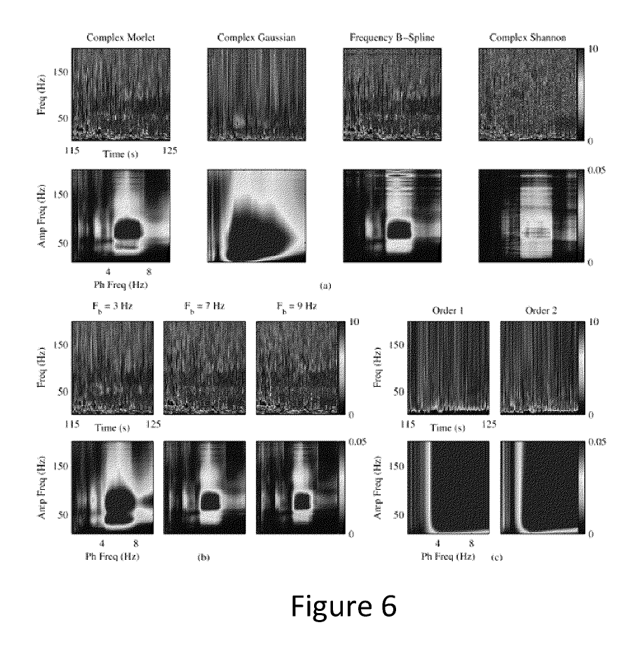

Four complex mother wavelets were investigated namely, complex Morlet, complex Gaussian, frequency b-spline, and complex Shannon. The complex wavelet coefficients of each were compared as well as the resulting MI. The Morlet wavelet with a bandwidth of 5 Hz was found to be the most useful when compared to other Morlet alternatives (FIG. 6B) while the Gaussian wavelet with an order of 5 was found to be the most useful in comparison to other Gaussian alternatives (FIG. 6C). In both cases, the spread in the wavelet was minimized while maintaining the integrity of the MI. When comparing all four complex mother wavelets, the Morlet was the most useful choice (FIG. 6A) and was thus used for the analysis.

FIG. 6A shows the wavelet coefficients of each mother wavelet in the first row. The complex Morlet had a bandwidth of Fb=5 Hz while the complex Gaussian had an order of 5. FIG. 6B shows three other bandwidths compared for the complex Morlet and FIG. 6C shows two other orders for the complex Gaussian. The versions selected for comparison with the other complex mother wavelets (as indicated in FIG. 6A) were found to the minimize the spread without compromising the integrity of the MI. Similarly, the complex Morlet with a bandwidth of Fb=5 Hz was found to be the most useful in comparison to the other three complex mother wavelets and was thus used for the subsequent analysis. Wavelet power was z-score normalized per frequency (in 1 Hz increments) using the mean and standard deviation of the wavelet power from a 30 s window more than 60 s before seizure onset. This allowed for all frequency activity to be visible on the same scale. MI was computed between the phase of the frequencies indicated on the x-axis and the amplitude of the frequencies indicated on the y-axis. All axes labels are as indicated on the leftmost panel and the scales are as indicated by the bar beside the rightmost panel on each of FIGS. 6A, 6B and 6C.

Prior to the pre-processing measures described above, two independent neurologists examined the recordings of each patient and identified channels involved in the seizure onset zones (SOZ). The identified channels were subsequently used for comparison with the ROI channels selected.

In accordance with step 110 of method 100, the degree of cross-frequency coupling (CFC) was measured by computing the MI [47], which is a measure of how the amplitude of a higher frequency (f_{H}) has a preference for the phase of a lower frequency (f_{L}). The time series of the amplitude envelope of f_{H}(i.e., A^{f}_{H}(t)) and the instantaneous phase of f_{L}(i.e., φ^{f}_{L}(t)) were extracted from the respective wavelet coefficients. A composite time series of the amplitude from f_{H} and the phase of f_{L} at each time point was then constructed [φ^{f}_{L}(t), A^{f}_{H}(t)]. Phases of f_{L} were binned in 20° intervals and the amplitude envelope of f_{H} within each bin j was averaged (i.e (A^{f}_{H})ⱼ). The mean amplitude was then normalized by the sum of all mean amplitudes in each phase bin, according to equation 2:

$$P ( j ) = \frac{{〈 {A}_{H}^{f} 〉}_{j}}{{\sum }_{k = 1}^{N} {〈 {A}_{H}^{f} 〉}_{k}} \qquad [ 2 ]$$

where N=18 for the number of phase bins, and the normalized amplitude P has discrete probability density function characteristics to namely, that P(j)≧0 for all j and the sum of P across all phase bins is unity.

The amplitude distribution was then compared to a normal distribution by measuring the Kullback-Leibler (KL) distance between the two distributions. The KL distance was normalized to make all values fall between 0 and 1. If there was no phase-amplitude coupling between f_{L} and f_{H} then the amplitude distribution resembled a uniform distribution, which was reflected in a normalized KL distance of zero. This normalized KL distance was effectively the MI and thus a larger distance between P and a uniform distribution was reflected in a larger MI value.

In this example, f_{L} was defined as 0.5 to 10 Hz in 0.1 Hz increments while f_{H} was defined as 11 to 450 Hz in 1 Hz increments. Complex wavelet coefficients were obtained using the Morlet mother wavelet with a bandwidth of 5 Hz and a center frequency of 0.8125 Hz. The MI was computed in 10 s windows that were shifted by 1 s. This allowed for five cycles of the lowest rhythm (i.e., 0.5 Hz) to be captured while maintaining a sufficient degree of continuity in the time domain. A 10 s window was also found to be the minimum window size required for reliably computing the MI [17].

Channels of interest were selected based on the initial appearance of strong MI as well as instances of sustained MI. Analogous to the 3 dB point of electronic amplifiers, strong MI was defined as ≧0.3 of the maximum MI value in each channel. The scale for each channel was set to 0.3 of the maximum MI value seen in that channel across all time. MI values exceeding this threshold were highlighted, thereby making it possible to visually compare the MI values across the grid. Depending on the patient, 2 to 4 channels were selected that exhibited consistent activity across all seizures obtained from that patient. These channels were then used as the center of a reduced 2-channel radius grid. Cross-channel MI was computed in this reduced grid for channel pairings with the center channel. Specifically, f_{H} was extracted from the center channel while f_{L} was extracted from the surrounding channels, up to a maximum of two channels away, and the MI was computed. This procedure was repeated with f_{L} being extracted from the center channel and f_{H} being extracted from the surrounding channels.

In order to determine if the modulation being observed was statistically significant, a method of surrogates was performed [46]. A surrogate time series was first created using the amplitude adjusted Fourier transform (AAFT). This method generated a vector of random numbers that follow a Gaussian distribution. The elements of this vector were then rank-ordered according to the same rank-order as the original time series. Jump discontinuities at the ends were suppressed by convolving the rank-ordered vector with a hamming window. The fast Fourier transform (FFT) was then applied to the output of this convolution and each phase was multiplied by e^{iφ}, where φ is a random number from a uniform distribution such that 0≦φ≦2π and φ(f)=−φ(−f). This requirement for symmetry allowed for the output of the inverse FFT (iFFT) to be a vector of only real values. The real-only output from the iFFT was the surrogate time series.

Once a surrogate time series was generated via AAFT, the complex wavelet coefficients thereof were computed for f_{L} (i.e., 0.5 to 10 Hz in 0.1 Hz increments). The complex wavelet coefficients for f_{H} of the original time series were then used in conjunction with these surrogate coefficients and a surrogate value for MI was computed (i.e., MIₛᵤᵣᵣ). The original MI (i.e., MI_{orig}) was compared to its surrogate counterpart and the instances of MI_{orig} exceeding MIₛᵤᵣᵣ were tabulated as this process was repeated N=200 times. A p-value for each pixel in the MI plot was computed from this tabulation. MI was considered significant if MI_{orig} exceeded MIₛᵤᵣᵣ for at least 95% of the surrogate cases. For comparison, p-values were also obtained by randomizing the amplitude rather than the phase of the signal (i.e., extracting the complex wavelet coefficients for f_{H} from the surrogate signal and f_{L} from the original signal) of a sample seizure. This was performed for the entire grid for a select frame and resulted in virtually identical p-values. The MI values were also z-score normalized for comparison using the mean and standard deviation from the distribution of surrogate MI values. The frequencies exhibiting normalized MI≧3 standard deviations above the mean were generally identical to those involved in significant MI as defined by the p-values described above.

FDR was then performed on the resulting p-values [6], [39] to control the rate of significant MI values. The p-values were first sorted in ascending order (pi, I=1 . . . N) then the maximum p-value was extracted which satisfied the criteria of equation 3:

$${p}_{i} < \frac{α i}{Nc ( N )} \qquad [ 3 ]$$

where α=0.05 and c(N)=1, since the p-values were positively correlated. This was set as the threshold for a p-value to be considered significant. Positive correlation in this context meant that a deviation from the null hypothesis by one electrode pair did not affect the deviations of the other electrode pairs.

Due to the computational expenses required by surrogate analysis, the method was applied to all channels in the 32-channel array for the three MI frames selected (i.e. at seizure onset, mid-seizure, and seizure termination). The onset and termination frames were selected such that they were within 30 s of the clinical onset and termination timestamps, respectively. The recordings for Patient D were longer than those of the other four patients so onset and termination frames were selected within 60 s of the clinical timestamps.

Global coherence (C_{global}) is a measure of the level of coordinated activity in a network. In order to determine the global coherence, the network first needs to be represented by a square matrix that can be eigenized. Once this eigenization is complete C_{global} is defined as the ratio of the largest eigenvalue to the sum of all eigenvalues, as shown in equation 4:

$${C}_{global} = \frac{{S}_{1}}{{\sum }_{i = 1}^{N} {S}_{i}} \qquad [ 4 ]$$

where Sᵢ is the i^{th} largest eigenvalue and N is the total number of eigenvalues. To form the square matrix prior to eigenization, the MI frames previously selected for surrogate analysis were used for computing the cross-channel MI for all 32×32 possible pairings. The mean MI for three select frequency ranges were obtained to namely, delta (0.5 to 4 Hz) modulating HFOs, theta (4 to 8 Hz) modulating HFOs, and all MI deemed significant via surrogates. HFO activity>200 Hz was only observed in Patient C and hence HFO was defined as 30 to 200 Hz for Patients A, B, D, and E and defined as 30 to 450 Hz for Patient C. These means resulted in three 32×32 square matrices for each of the three select MI frames. Each matrix was then eigenized and the eigenvector associated with the largest eigenvalue was extracted. Each component of the eigenvector represents how each channel is contributing to the eigenvector. As such, the mean across the thirty-two (32) components of the eigenvector was obtained and a threshold was set to three standard errors of mean above the mean. Any components above this threshold were used to select the corresponding channels as channels within the ROIs.

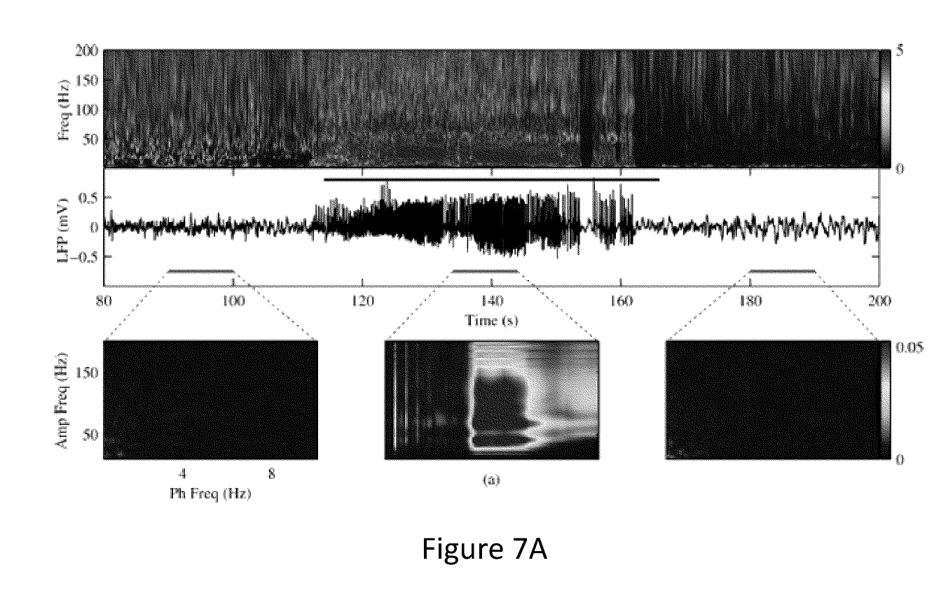

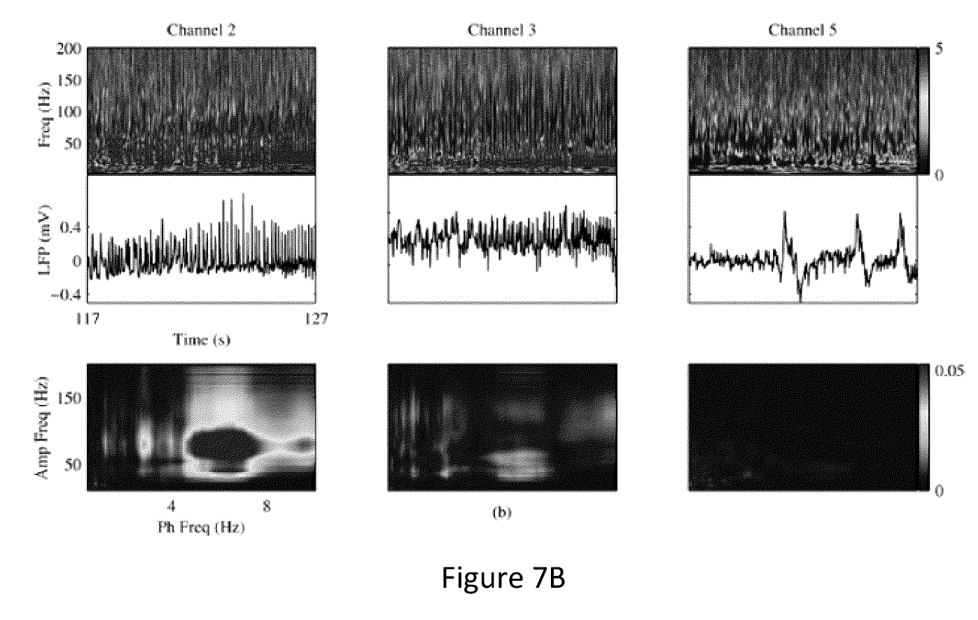

Seizures in Patients A, B, C and D were observed as high amplitude bursts in the local field potential (LFP) relative to the non-seizure time segments. The seizures of the Patient E were difficult to identify in the electrographic activity, as there were no observable changes in the time domain of the LFP. In accordance with step 130 of method 100, FIG. 7A illustrates a typical seizure from Patient A. Both the LFOs and HFOs coincide with the seizure duration and were present simultaneously. Hence, modulation of the HFOs by the LFOs was observed only during the seizure. The large horizontal line indicates clinical seizure while the three smaller horizontal lines indicate the 10 s windows for which the MI values were computed. In the top panel of FIG. 7A, the wavelet coefficients are shown. As can be seen, strong MI was observed during the seizure but not during non-seizure activity.

The presence of the LFOs and HFOs was not restricted, however, to co-existence (FIG. 7B). HFOs were observed in the absence of the LFOs in some channels and similarly, LFOs were also observed in the absence of HFOs in others. In accordance with step 140 of method 100 and as shown in FIG. 7B, channel 2 has both HFO and LFO activity present while channels 3 and 5 were dominated by LFO and HFO activity, respectively, and hence, MI was more strongly observed in Channel 2 during this time window, thereby suggesting spatial specificity, wavelet power was z-score normalized per frequency (in 1 Hz increments) using the mean and standard deviation of the wavelet power from a 30 s window more than 60 s before seizure onset, allowing all frequency activity to be visible on the same scale, the MI being computed between the phase of the frequencies indicated on the x-axis and the amplitude of the frequencies indicated on the y-axis.

For Patient A, modulation was observed only during the clinical seizure and was characteristic of only select channels. For Patient E, the modulation was restricted to specific channels, this LFO-HFO modulation was observed irrespective of the clinical seizure. For Patient E, modulation was observed during both seizure and non-seizure activity. For Patients B and C, the modulating LFO was consistently the delta rhythm, while for Patient E it was consistently the theta rhythm. For Patients A and D, the LFO was initially the theta rhythm then shifted into the delta rhythm as the seizures developed. Rhythmic delta activity has been previously found to be the LFO with the highest prevalence at seizure onset in ETLE at a rate of 24% compared to theta activity at 8% [21]. In TLE, theta is present at seizure onset at a rate of 45% while delta is slightly lower at 35%. The modulated HFOs were found to vary depending on the patient, as will be discussed.

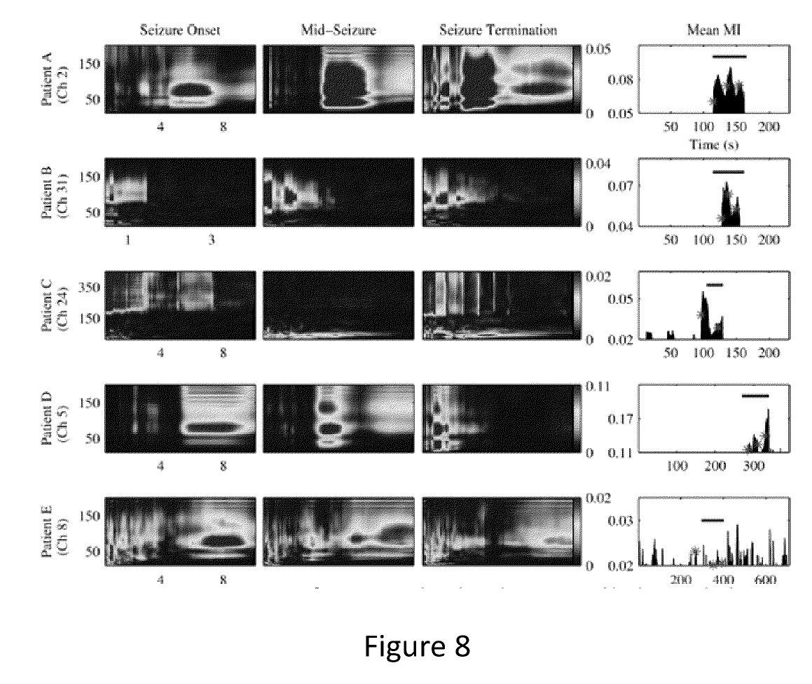

HFO modulation by the LFOs was observed in select channels of the grid. Seizure onset and termination was marked by the first and last appearance of strong modulation, respectively, for Patients A, B, C and D. The modulation is illustrated in FIG. 8.

In FIG. 8, the MI in a single channel from these frames is shown for each patient across each row. The MI was computed between the phase of the frequencies indicated on the x-axis and the amplitude of the frequencies indicated on the y-axis. For Patient B, the MI was restricted to delta modulation and hence, the phase frequencies on the x-axis are set as 0.5 to 4 Hz. For Patient C, the onset and termination HFOs are above the ripple range and hence, the amplitude frequencies on the y-axis were set as 11 to 450 Hz. The mid-seizure frame for Patient C shows activity around the 40 Hz rhythm while the higher frequencies were restricted to seizure onset and termination. For Patient A, D and E, the phase frequencies were set as 0.5 to 10 Hz and the amplitude frequencies were set as 11 to 200 Hz. Modulation was observed by both the delta and theta rhythms for Patient A and D while it was restricted to the theta rhythm for Patient E. The mean MI of the values above the scale maximum is shown on the right side of each respective row. The horizontal bar indicates the clinical seizure while the asterisks indicate the time of the MI window in the panels on the left in order of appearance. For Patients A, B, C and D, the temporal specificity of MI is emphasized. Patient C shows some sporadic MI activity prior to the seizure. The mean MI of the mid-seizure frame for Patient C is below the scale maximum and hence is not on the respective plot (the asterisks indicate the mean MI from the onset and termination frames). As can be seen, the modulation of Patient E is more restricted in space than in time.

For Patient A, strong MI was observed at seizure onset with the modulating frequency being predominantly the theta rhythm. As the seizure progressed in time, the modulating frequency shifted towards delta. The modulated rhythm at seizure onset was centered around 80 Hz and the range of modulated frequencies widened as the seizure progressed in time, at its widest encompassing 30 to 200 Hz. As the seizure approached its termination, this range also decreased as was observed during onset. Outside of the seizure time, modulation was not observed. Some studies have reported gamma (30 to 100 Hz) and ripple (110 to 160 Hz) oscillations being nested within the same theta cycle [48]. Specifically, the amplitudes of these HFOs peaked at different theta phases and hence modulation of this wide frequency range was observed.

For Patient B, modulation prior to the seizure was observed in select channels for one of the two recorded seizures. However, once the seizure onset was reached, the channels exhibiting modulation prior to the seizure ceased doing so. Moreover, select channels that were dormant prior to the seizure began to exhibit strong modulation and continued to do so until seizure termination. These select channels were consistent for both seizures of Patient B. Once seizure termination was reached, modulation was not observed in any of the channels. Unlike Patient A, the modulating frequency remained within the delta rhythm range for the entirety of the activity. Similarly, the HFO being modulated was centered around the 80 Hz rhythm and the range did not expand or contract as the seizure developed.

Modulation from Patient C was unique from the other four patients in that the HFO being modulated was the fast ripple (i.e., 200 to 450 Hz). The modulating LFO was the delta rhythm. Fast ripple modulation was not observed in any other patient. This modulation was also only present at seizure onset and termination and was dormant for the duration of the seizure itself. This phenomenon was observed in all channels across the grid. However, delta and theta modulation of the 40 Hz rhythm was observed in select channels for the duration of the seizure. The first (last) instance of this modulation were slightly delayed (premature) of the seizure onset (termination) and consequently of the associated delta-fast ripple modulation as well.

Patient D was somewhat similar to Patient A in that the modulating frequency was initially theta, which was observed at seizure onset, and as the seizure developed this modulating frequency shifted to the delta rhythm. The HFOs being modulated were the rhythm centered around 40 Hz and frequencies>140 Hz. The strength of the modulation of these HFOs varied as the seizure progressed. Once seizure termination was reached, the modulating frequency was the delta rhythm.

The seizures of Patient E were classified as atypical by the neurologist and they did not always manifest in the form of convulsions, which made them more difficult to identify in time. This was evident in the modulation as well. The modulating frequency remained stationary at the theta rhythm and the HFO being modulated was consistently centered around 80 Hz. This modulation, however, was not restricted to the time duration identified as the clinical seizure. The timestamps defining seizure onset and termination were based on the neurologist's notes and were accompanied by convulsions

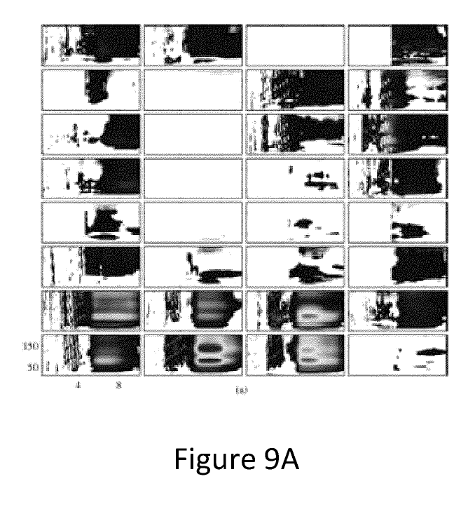

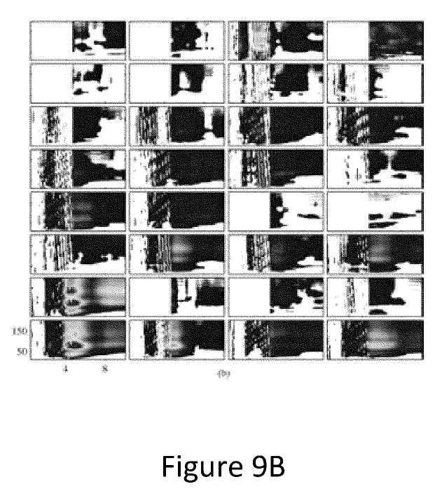

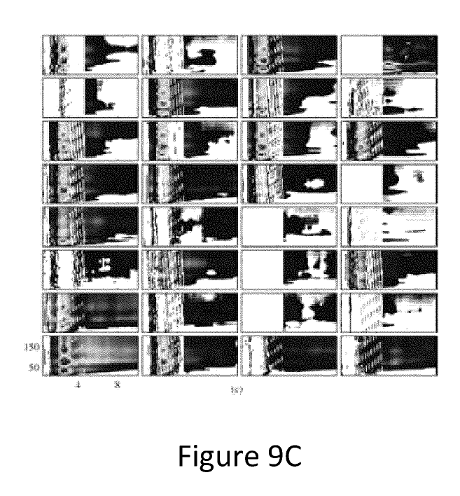

The modulation identifying seizure onset and termination, as described above, was observed only in select channels. This modulation also spread across the grid in characteristic patient-specific patterns as the seizure developed in time. Visual inspection of the modulation as it progressed in time was used to identify channels with strong MI. EVD was also used to identify channels defining the ROI(s). An illustrative example of the seizure onset, mid-seizure, and seizure termination frames of the grid for Patient D is shown in FIGS. 9A, 9B and 9C, respectively. These frames were used for computing the cross-channel MI across the entire grid from which the mean MI of select frequencies was obtained.

In accordance with step 110 of method 100, FIGS. 9A, 9B and 9C show boxes containing the MI values computed in the same lOs window across all thirty-two (32) channels of the reduced grid. MI was computed between the phase of the frequencies indicated on the x-axis (0.5 to 10 Hz) and the amplitude of the frequencies indicated on the y-axis (11 to 200 Hz). The scale for each channel was set as zero to the 30% of the maximum MI value observed in that channel across all frames, which is analogous to the 3 dB point. Surrogate analysis was performed on each of these frames and FDR was subsequently applied. White indicates MI values that were not found to be significant (p>0.05). MI is first observed in channels 2, 3, and 7, where the modulating LFO was the theta rhythm. As the seizure progressed, this LFO shifted towards the delta rhythm, as seen in the mid-seizure frame. The shift towards delta-modulation was also the beginning of modulation in the upper region of the grid, specifically channel 31. Once the seizure neared its termination, this delta-modulation dominated the upper region of the grid, as seen in the seizure termination frame. This suggests that Patient D has two ROIs: the first in the lower region of the grid when theta is the dominating LFO and the second in the upper region of the grid when the dominating LFO is the delta rhythm. Note that the lower part of the grid also had a delta dominating LFO, which would have extended the ROI estimated from the theta dominating LFO to that is, channels land 5 in addition to channels 2, 3, and 7.

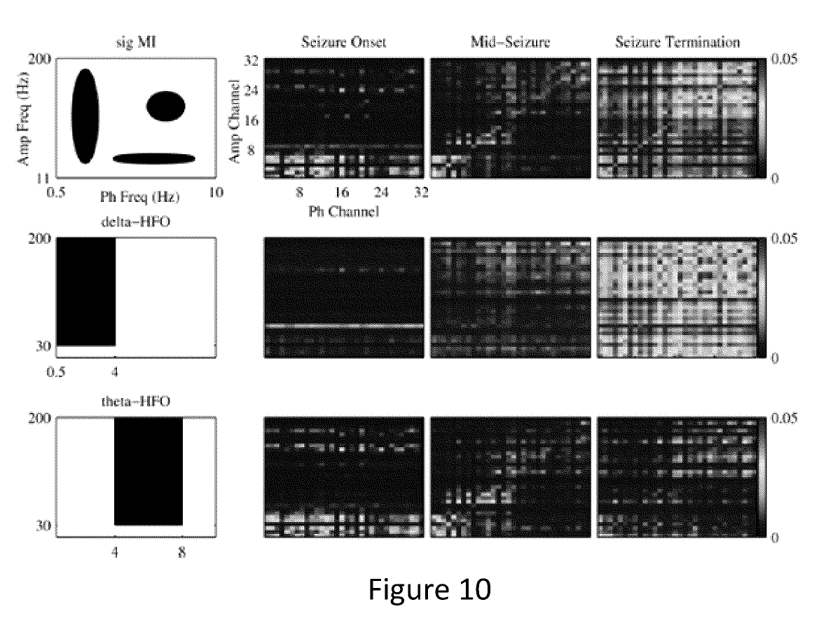

An illustrative example of the resulting square matrices of mean MI values is shown in FIG. 10. In accordance with step 120 of method 100, the mean MI is obtained for all MI values that are found to be significant (first row), all MI values within the delta-HFO frequencies (second row), and all MI values within the theta-HFO frequencies (third row). The frequencies are illustrated on each respective row. The channel from which the phase of the LFO is extracted is indicated on the x-axis while the channel from which the amplitude of the HFO is extracted is indicated on the y-axis. At seizure onset, the lower left corner of the matrix was more active when the mean is from the theta-HFO modulation whereas at seizure termination the upper right corner of the matrix was more active with the delta-HFO modulation. The mean significant MI was active at both seizure onset and termination in the lower left corner and upper right corner, respectively, but with lower means. Each matrix was z-score normalized using the mean and standard deviation to allow for appropriate comparison between pairings.

During seizure onset for Patient D, the modulating LFO was predominantly the theta rhythm and hence the matrix of theta-HFO modulation showed higher activity. Moreover, this modulation was seen in a horizontal rectangle in the bottom left corner suggesting that the lower numbered channels were more involved in the seizure onset than the rest of the grid and this involvement was predominantly theta-modulated HFOs. During seizure termination, the upper right corner of the delta-HFO modulation matrix was more active suggesting that the delta rhythm was the modulating LFO and also that the higher numbered channels were involved in this modulation more than the rest of the grid. The location of these rectangles (i.e., the lower left corner followed by the upper right corner) suggest that cross-channel modulation was stronger in channels of close proximity compared to the modulation involving channels across the grid, which would have manifested as stronger activity in the upper left and lower right corners of these matrices. Also, the horizontal nature of these rectangles further suggests that selection of the channel for LFO extraction was not as significant as selection of the channel for HFO extraction. This was also observed when the cross-channel modulation was computed in the reduced grids centered around the channels selected by visual inspection of MI.

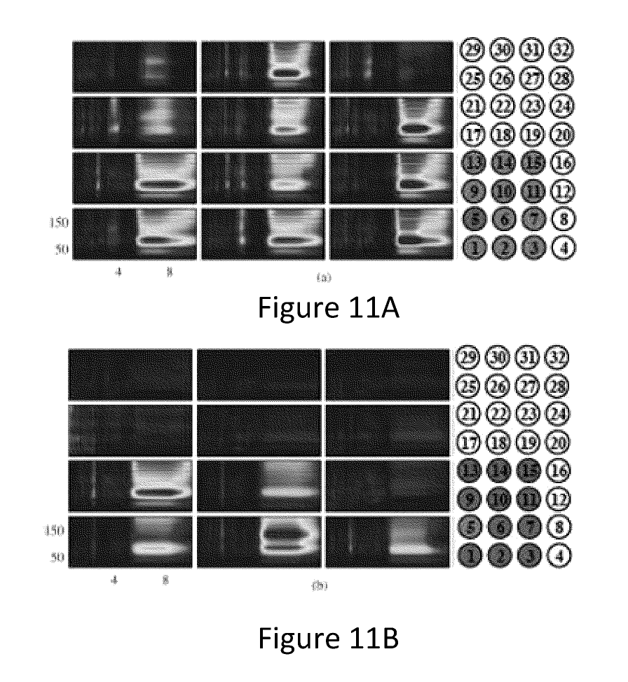

FIGS. 11A and 11B illustrate how the area defined by the modulation between the HFOs of the center channel and the LFOs of the surrounding channels was larger than that defined by the modulation between the center channel LFOs and the HFOs from the surrounding channels. Cross-channel modulation was computed in reduced grids with a 2-channel radius around a center channel across all time. The center channels were the channels selected by visual inspection of the MI grids. For Patient D, channel 5 was one of the selected channels. For the same 10 s window, cross-channel modulation was computed by extracting the HFOs from channel 5 and the LFOs from the surrounding 2-channel radius and by extracting the LFOs from channel 5 and the HFOs from the surrounding 2-channel radius. The MI was computed between the phase of the frequencies indicated on the x-axis (0.5 to 10 Hz) and the amplitude of the frequencies indicated on the y-axis (11 to 200 Hz). The scale for each channel was set as 0 to 0.3 of the maximum MI value observed in that channel across all frames, which is analogous to the 3 dB point.

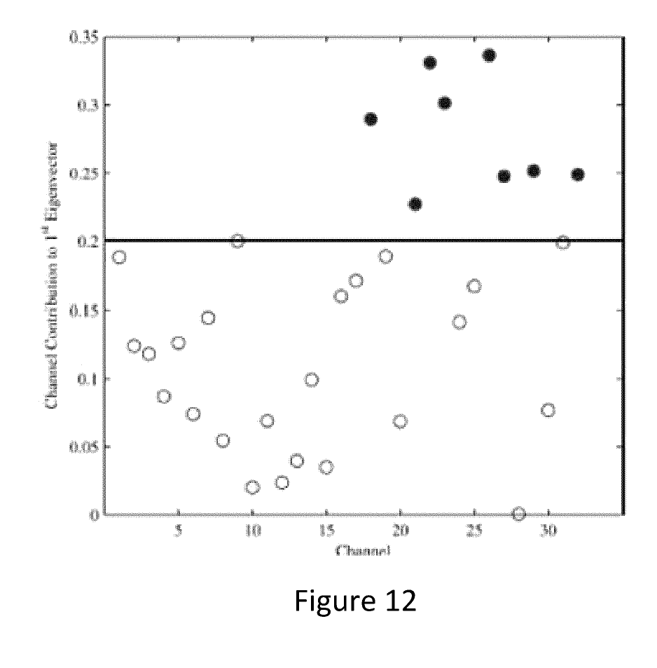

EVD performed on these matrices yielded thirty-two (32) eigenvectors associated with thirty-two (32) eigenvalues and the contribution of each channel to the eigenvector associated with the largest eigenvalue was used as the basis of channel selection, as shown in FIG. 12.

In accordance with step 120 of method 100, in FIG. 12, the eigenvector associated with the largest eigenvalue is shown. The mean of all thirty-two components of the eigenvector was obtained and the threshold was set to three standard errors of mean above the mean (indicated by the horizontal line). Components of this eigenvector above this threshold are indicated and correspond to the channel numbers on the grid. As will be appreciated, the EVD-selected channels were selected in this manner.

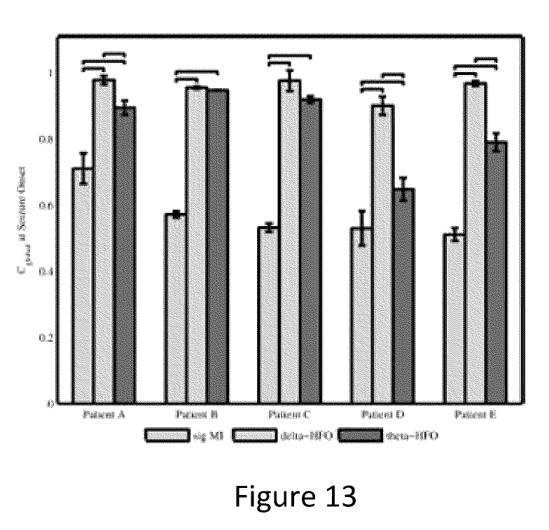

The C_{global} computed from the eigenvalues for each configuration of mean MI during seizure onset is illustrated in FIG. 13. C_{global} was computed from the eigenvalues extracted from the matrices of the mean MI values. At seizure onset, C_{global} was found to be significantly lower when computed from the mean significant MI values in all patients. The mean delta-HFO modulation results in higher C_{global} compared to mean theta-HFO modulation for Patient A, D, and E. The horizontal lines indicate significant differences (p<0.1).

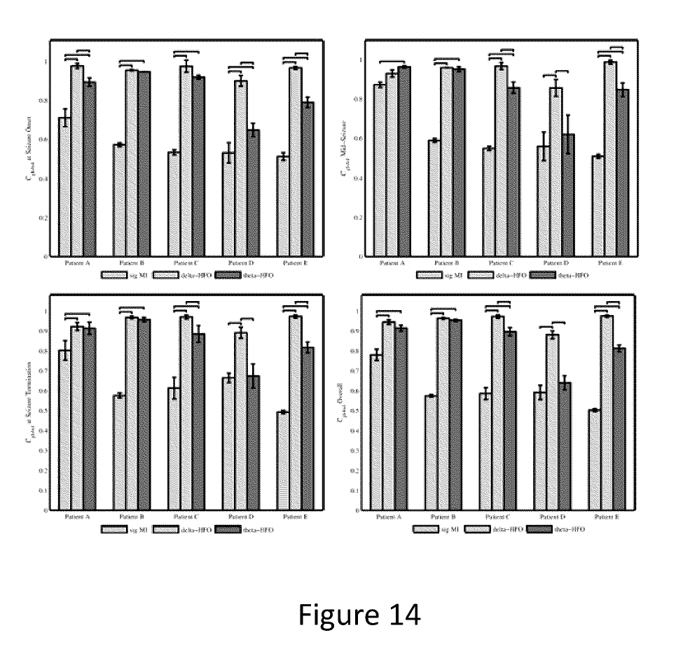

C_{global} was the lowest when computed from the mean significant MI in all five patients. Delta-HFO modulation resulted in the highest C_{global} in Patients D and E and was not significantly different from that computed by theta-HFO modulation for Patients B and C. The C_{global} computed from the mid-seizure and seizure termination frames as well as the cumulative summary of all three frames shown in FIG. 14.

In FIG. 14, C_{global} was computed from the eigenvalues extracted from the matrices of mean MI values at seizure onset (top left panel), mid-seizure (top right panel), seizure termination (bottom left panel), and the overall cumulative summary of all three frames (bottom right panel). C_{global} was found to be significantly lower when computed from the mean significant MI values in all patients across all frames as well as the overall summary. Mean delta-HFO modulation resulted in higher C_{global} compared to mean theta-HFO modulation for Patient A, D, and E during seizure onset and for Patient C mid-seizure, seizure termination, and in the overall cumulative summary. For Patient B, delta-HFO and theta-HFO modulation resulted in comparable C_{global} values. Horizontal lines indicate significant differences (p<0.1);

For Patient C, delta-HFO modulation resulted in a higher value of C_{global} compared to that of theta-HFO modulation in the overall cumulative summary of all frames.

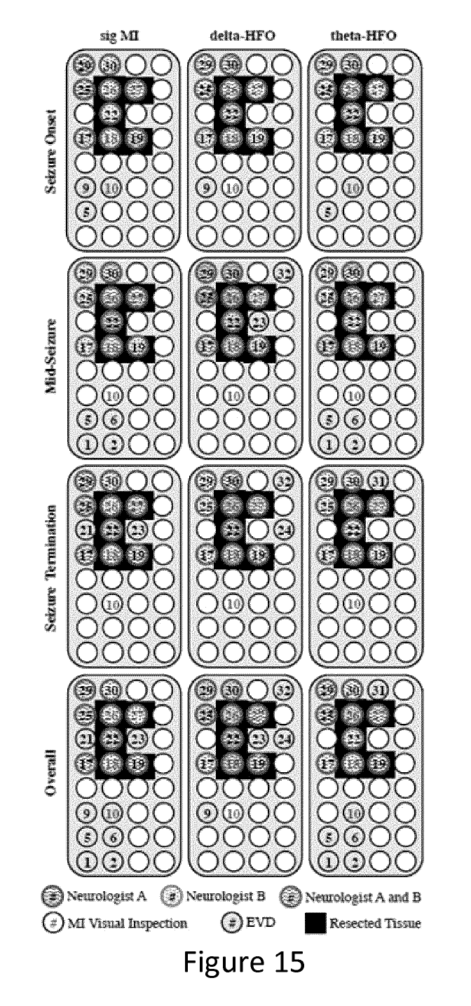

The ROIs identified by both methods, namely, visual inspection of MI progression in time and EVD performed on the seizure onset frame, are summarized and compared to the SOZs defined by two independent neurologists in FIG. 15. Specifically, the channels defining the ROIs were identified from all three MI frames for Patient D and the overall cumulative summary of the identified channels are shown. At seizure onset, the ROI identified is in the lower left corner of the grid and is more largely defined by the significant MI and theta-HFO modulation. Mid-seizure, the delta-HFO modulation identifies an ROI in the upper region of the grid while the theta-HFO modulation identifies the lower left corner of the grid. At seizure termination, the LFO is predominantly the delta rhythm and this is reflected in the delta-HFO modulation identifying the upper region of the grid, where the delta was more active. The overall cumulative summary of the identified ROIs highlights both regions on the grid: the upper half by delta-HFO modulation and the lower left corner by theta-HFO modulation. The SOZ identified by Neurologist A is partially resected due to its overlap with eloquent cortex. Resected tissue is shown as a dark region on the grid. Patient D is classified as Engel Class III. The same channel may be selected by multiple methods;

For Patient D, the channels identified during seizure onset were not sufficient to identify the ROIs and hence the mid-seizure and seizure termination frames were also investigated (FIG. 15). It is noted that neurosurgeons generally resect tissue slightly outside of the defined region to minimize the risk of a secondary surgery. Differential electrodes were obtained by taking the difference between adjacent horizontal channels effectively widening the distance between the channels in the horizontal plane of the grid. However, the distance between adjacent vertical channels remained unchanged. Thus a one-channel boundary around each neurologist-selected channel in the vertical plane was considered as part of the identified SOZ. Surgical resection (corticectomy) was performed based on the channels identified by Neurologist A.

The initial instance of modulation for Patient A was strongest in channel 2 and was also visible in channels 3, 8, 12, 14, 25, and 29. At its point of widest spread, modulation was observed in the channels immediately adjacent to these initial channels. Visual inspection identified channel 2, which is adjacent to

the channel selected by Neurologist B, and channel 12, which was one of the channels identified by Neurologist A. Additionally, channels 25 and 29 were identified by visual inspection but missed by both neurologists. This patient did not undergo resection surgery due to the SOZ being located on eloquent cortex. This region was also found to have a lesion.

The modulation for Patient B was seen outside of the seizure times in one of the two recorded seizures from this patient. This modulation was observed in channels 3, 10, 11, 15, 26, 29, and 30. In this patient's second seizure this pre-seizure modulation was not observed. Once the seizure onset was reached, however, strong modulation was seen only in channels 27, 28, 31, and 32 and slightly weaker in channels 20 and 24 for both seizures. After seizure termination, modulation was not observed in any of the channels in either seizure. Neurologist B identified channels most similar to those identified by EVD performed on delta-HFO modulation and significant MI. The channels identified by Neurologist A, however, were closest to those identified by theta-gamma mean MI, which also identified the largest region on the grid. This patient had the worst post-surgical outcome and was classified as Engel Class IV, which is reflected in the numerous EVD-selected channels that were not identified by either neurologist.

Modulation for Patient C was seen across the entire grid in all 32 channels at seizure onset. As previously mentioned, the modulating frequency for this patient remained within the delta rhythm. For one of the two seizures, the HFO being modulated was the ripple (i.e., 80 to 200 Hz) while for the second seizure the HFO was the fast ripple (>200 Hz) at seizure onset and termination. The rhythm centered on 40 Hz was also being modulated by the upper delta and lower theta rhythm (3 to 5 Hz). However, this modulation was only observed in channels 20 and 24, which were ultimately selected by EVD with both delta- and theta-modulated HFOs. Both neurologists were in agreement about identifying channel 24. Moreover, the remaining channels identified were adjacent. Patient C had an improved post-surgical outcome and was classified as Engel Class II. This was also reflected in the fewer number of EVD-selected channels that were not identified by the neurologists and hence were not resected.

Patient D had modulation first appearing in channels 2, 3, and 7, where the modulating frequency was the theta rhythm. As the seizure developed, this modulation also appeared in channel 5. EVD performed on the seizure onset frame selected channels in the lower left corner of the grid, with the mean significant MI and theta-modulated HFOs identifying a slightly larger area than delta-modulated HFOs. This is consistent with the higher activity seen in the lower left corner of the corresponding MI matrices in FIG. 10. The modulating frequency shifted into the delta rhythm as the seizure continued to develop. Once the delta rhythm was involved in the modulation, strong modulation was also observed in the upper region of the grid to namely, starting in channel 31 and spreading to the upper half of the grid. This transition is evident in the MI frames illustrated in FIGS. 9A, 9B and 9C, and is seen in the upper right corner of the delta-modulated MI matrix in FIG. 10. Both neurologists identified channels 18 and 22. Neurologist A identified additional channels in the upper region of the grid, which was dominated by delta-modulation. This patient had a limited resection, again, due to the SOZ being on eloquent cortex and was classified as Engel Class III.

Although the modulation for Patient E was observed irrespective of the clinical seizures, it was observed only in select channels to namely, channels 8, 16, 28, and 32, with channel 8 exhibiting the strongest and most sustained modulation. Modulation was not observed in the remaining channels of the grid. Neurologist A identified channel 8 while Neurologist B did not identify any channels, suggesting that the SOZ was located outside the boundaries of the grid. This patient underwent resection surgery and was classified as Engel Class II. This outcome was reflected in the fewer number of channels that were identified by EVD and visual inspection of MI but not identified by either neurologist and hence were not resected, as was the case with Patient C.

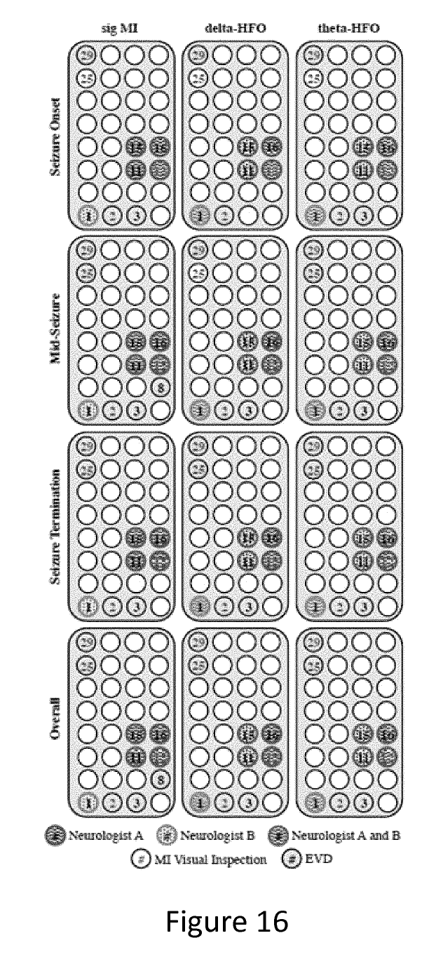

For Patients A, C, and E, delta- and theta-HFO modulation as well as mean significant MI resulted in similar channel selections by EVD. For Patient B, theta-HFO modulation resulted in a larger region selected on the grid. An illustrative example of the EVD channel selection based on each MI frame to namely, seizure onset, mid-seizure, and seizure termination to is shown in FIG. 15 for Patient D and FIGS. 16 to 19 for the remaining four patients. In accordance with step 120 of method 100, channels were identified in at least two of the three frames. For three patients a maximum of two channels were identified in only one of three frames to namely, for Patient A channel 1 was identified during termination and channel 8 mid-seizure, for Patient C channels 2 and 10 were identified at termination, and for Patient E channel 12 was identified during onset while channel 32 mid-seizure.

For Patient A, all channels identified in the seizure onset frame were also identified in the mid-seizure and seizure termination frames (FIG. 16). Additionally, channels 3 and 25 were selected in the two latter frames but not in the seizure onset frame. There was no significant different in the identified channels over the three frames. Patient A did not undergo resection surgery.

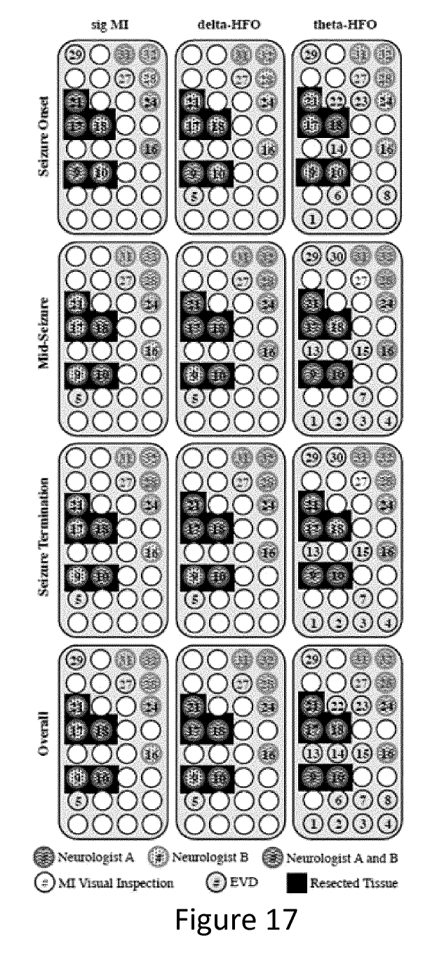

For Patient B, a total of nineteen channels were identified across all three frames using the theta-HFO modulation, eight of which were identified only in the seizure onset frame to namely, channels, 6, 8, 9, 14, 18, 22, 23, and 27 (FIG. 17). Eleven channels were consistently identified in all three frames. There was no significant difference in the identified channels over the three frames for the significant MI and delta-HFO modulation schemes. The theta-HFO modulation identified additional channels mid-seizure and at seizure termination in the lower half of the grid. Resected tissue is shown as a dark region on the grid. Patient B was classified as Engel Class IV.

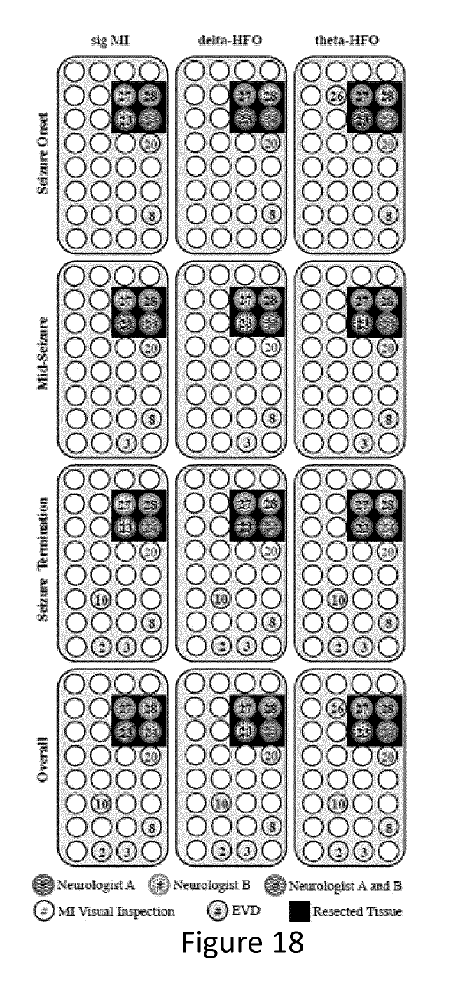

For Patient C, channels 26, 27, and 28 were identified only in the seizure onset frame while channels 3 and 8 were only identified in the mid-seizure and seizure termination frames (FIG. 18). The mid-seizure and seizure termination frames identified three additional channels in the lower half of the grid. Resected tissue is shown as a dark region on the grid. Patient C was classified as Engel Class II.

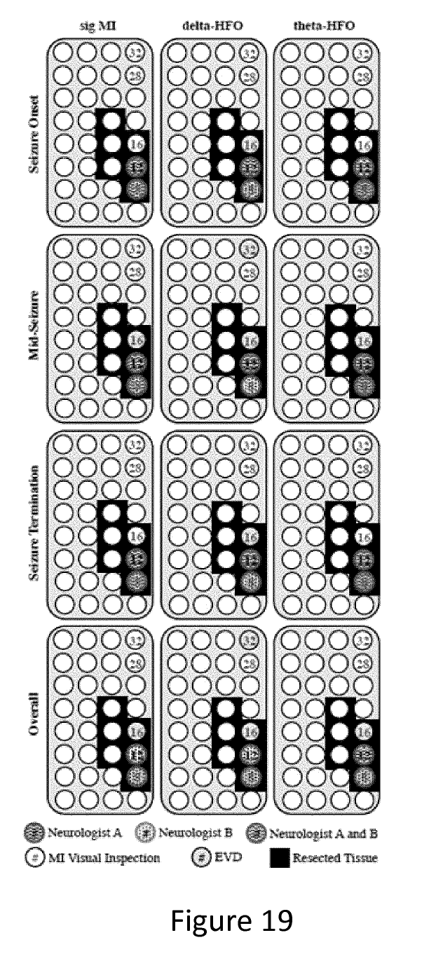

For Patient E, channel 8 was consistently identified in all three frames using theta-HFO modulation and channel 16 using delta-HFO modulation (FIG. 19). Channel 32 was only identified in the mid-seizure frame using delta-HFO modulation. There was no significant difference in the identified channels over the three frames. Resected tissue is shown as a dark region on the grid. Patient E was classified as Engel Class II.

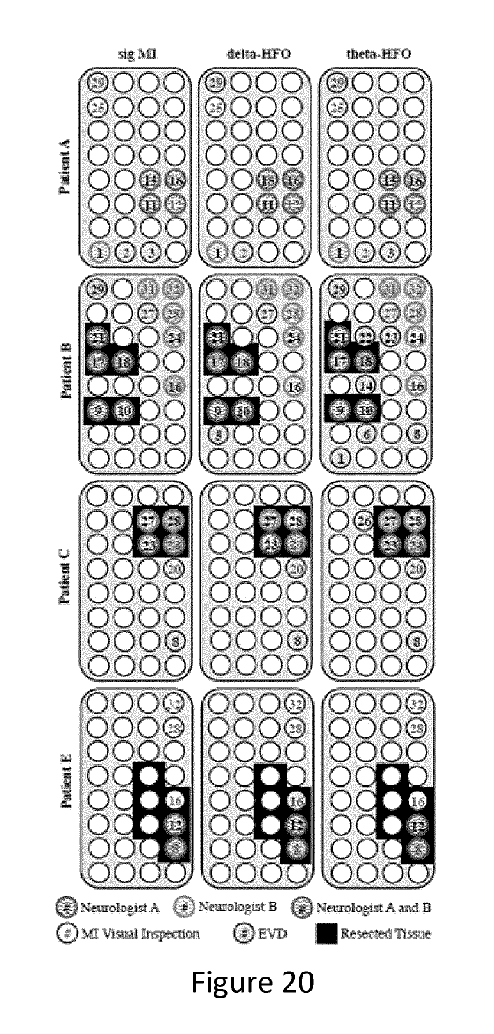

For Patients A, B, C and E, almost all the identified channels were found either only in the seizure onset frame or with some consistency in all three frames. Few channels were identified apart from the time of seizure onset, which suggests that the involvement of each of these multiple ROIs was manifested either at the beginning of the seizure or throughout its entirety. The ROIs identified based on the seizure onset for Patients A, B, C, and E are summarized in FIG. 20. For Patient B, theta-HFO modulation identified a larger ROI on the grid whereas the ROIs identified for the other patients (Patients A, C and E) did not differ between the three modulation schemes. Surgical resection (corticectomy) was based on the channels identified by Neurologist A (green) with the exception of Patient A (no resection was performed due to the SOZ being located on eloquent cortex). Resected tissue is shown on the grid. Neurologist B did not identify any SOZ channels for Patient E suggesting it was located outside the boundaries of the grid.

For Patient D, however, a second ROI was identified in the latter half of the seizures (FIG. 15). At seizure onset, the theta-modulated HFOs were able to identify channels in the lower left corner of the grid to namely, channels 5 and 10 to while the delta-modulated HFOs only identified channel 9. Mid-seizure, the theta-HFO modulation additionally to identified channels 2 and 6 while delta-HFO modulation were able to identify the upper region of the grid, which included the channels selected by both neurologists to namely, channels 18, 22, 23, 25, 26, 29, 30, and 32. As seizure termination was reached, the theta-modulated HFOs only identified channels 26 and 31 while the delta-modulated HFOs identified channels 18, 22, 24, 26, and 32. This was consistent with how the MI developed in time when modulation was observed between HFOs and LFOs extracted from the same channel, as previously illustrated in FIGS. 9A, 9B and 9C, as well as what was seen in the cross-channel MI matrices (FIG. 10). Essentially, the first ROI for Patient D is defined by theta-modulated HFOs in the lower left region of the grid. Once the modulating LFO becomes the delta rhythm, the second ROI in the upper left region of the grid becomes active and the seizure continues in this manner until its termination.

The HFO rhythms being modulated by the LFOs varied between patients. One explanation for this variation is the difference in the cognitive state among the patients. Specifically, the sleep stage of the patient has previously been found to have an effect on HFO properties [4], [15]. During non-REM sleep, HFOs were found to be the fastest when compared to REM sleep and wakefulness. All three seizures of Patient A were recorded during wakefulness while both seizures of Patient B and both seizures of Patient C were recorded during sleep. One of the three seizures of Patient D was recorded during wakefulness while the remaining two were during sleep. For Patient E, it was difficult to determine whether or not she was sleeping, as she was resting in bed during all five seizures but may not have necessarily been asleep. No attempts in this work were made to determine the sleep stage at the time prior to the seizures for any of the patients. However, the effect of sleep stage may be a contributor to the patient-specific nature of the HFO being modulated. Moreover, sleep stage was found to have an effect on the spatial specificity of fast ripples [4]. Specifically, HFOs recorded during non-REM sleep have been suggested to be more indicative of the SOZ than those recorded during REM or wakefulness. One of the seizures of Patient B exhibited strong modulation in select channels outside of the ROI prior to the seizure, which then became dormant during the seizure itself. This activity was not observed in the patient's other seizure. This may be due to the patient being in a different sleep stage in the two seizures.

Observing the seizures in the MI domain rather than in the time or frequency domains identified multiple ROIs. In general, the channels identified by visual inspection of MI provided a more conservative selection of channels compared to those identified by EVD. However, EVD did capture most of the channels identified by visual inspection in all five patients. For Patient A, Neurologist A identified a lesion in channels 11, 12, 15, and 16 in a magnetic resonance image and thus inferred that this lesion may coincide with the SOZ. Neurologist B only examined the electrographic recordings and was the closest to identifying the same channels as EVD. Neurologist B indicated that he often selects channels by identifying the most active point of the seizure and examining the delta rhythm and ripples, if present, from that point moving backwards towards onset. Hence, examining not only the seizure onset frame but also the mid-seizure and seizure termination frames to facilitate channel selection was warranted. For four of the five patients, examining the seizure onset frame was sufficient to identify the ROIs. However, the unique nature of the seizures of Patient D required further examination of both the mid-seizure and seizure termination frames. Similarly for Patient B, Neurologist B and EVD channel selection were in most agreement with the delta-modulated HFOs while for Patient C channel selection was not dependent on the LFO involved in the modulation. For Patient D, EVD selected the same combined channels of Neurologist A and B for delta-modulated HFOs mid-seizure but not for the theta-modulated HFOs. During seizure termination, the EVD-selected channels were most similar to those selected by Neurologist B when looking at the delta-modulated HFOs. The LFO of Patient E was consistently the theta rhythm and did not shift into the delta rhythm at any point during the seizure or non-seizure segments of the recording. Consequently, Neurologist B did not identify any channels on the grid as SOZ channels.

The ROIs identified for each patient included additional channels to those defining the SOZ for resection identified by Neurologist A and also those identified by Neurologist B. Moreover, the post-surgical outcome of each patient was correlated to the number of additional channels that were not identified by either neurologist. Specifically, a poorer surgical outcome was observed in patients with more EVD selected channels that were not identified by the neurologists. Patient B had the worst outcome, being classified as Engel Class IV, and also had the highest number of unidentified channels by the neurologists. Patient D, who was classified as Engel Class III, had fewer unidentified channels and was followed by Patients C and E, both of which were classified as Engel Class II. None of the patients had all EVD selected channels resected and, consequently, none of the patients were classified as Engel Class I.

Two studies have looked at CFC between HFOs and varying LFOs, both of which used children with subdural grids as subjects. One study examined the seizures of seventeen (17) children with focal epilepsy secondary to focal cortical dysplasia and compared the MI values from the SOZ, early propagation zone, and non-epileptic cortex of both seizure and non-seizure segments [24]. Raw data was bandpass filtered into conventional bands, starting with the delta rhythm up to and including the fast ripple with a maximum frequency of 300 Hz, after which the Hilbert transform was applied and the amplitude envelope of each band and the instantaneous phase were extracted. These were then used for computing the corresponding MI values [12]. They found significantly elevated CFC within the SOZ during the seizure but not during non-seizure segments and no significant CFC during either the seizure or non-seizure segments in the early propagation zone or non-epileptic cortex. Moreover, their results suggest a high specificity (79-100%) for identifying the SOZ but low sensitivity. The neurologist-identified area was larger and more numerous than that identified by elevated CFC. This study, however, did not indicate at which point during the seizure the reported results were observed or if the results were the average over all time points during the seizure. If it was a single time point, it is not indicated if the same time point was used when comparing the CFC occurring at the SOZ compared to the other two regions. It was not stated if the spatial and temporal characteristics of the seizure activity in this study were investigated separately. During seizure onset, significant MI was also only observed in select channels. As the seizure reached termination the modulation spread to the entire grid, after which it was no longer present. Studies have found that the LFO involved in seizure CFC was predominantly the alpha rhythm when compared to the theta rhythm [24]. Specifically, at seizure termination, the HFO amplitudes were maximal at the alpha phase trough but were inconsistent at seizure onset. However, when the modulating LFO was delta, the HFO amplitudes were maximal at the delta phase peak regardless of the seizure progression. In the above example, delta-modulated HFOs were observed in four of five patients.

Other studies investigated if HFOs (80 to 200 Hz) during the seizure were coupled with the phase of slow-waves, if these slow-waves were locally synchronous or globally, and if coupling between HFO amplitudes and slow-wave phases differed between seizure and non-seizure states [38]. They looked at the seizures of eleven (11) children and found that HFOs were tightly locked to the phase of the slow-wave at ≦1 Hz during the seizure. These slow-waves were found to propagate from the SOZ to surrounding regions. In the above example, the cross-channel modulation computed for the reduced grid with the HFOs extracted from a center channel and the LFOs extracted from the surrounding channels was found to spread across a larger area of the grid compared to the modulation of the LFO-centered reduced grid. The modulating LFO was either delta or theta, depending on the patient and the progression of the seizure. The spread of the LFO from the ROI is similar to the propagation of the slow-wave from the SOZ described in children. The difference in the LFO may be due to the age difference between the two patient populations. The study also found that non-seizure HFOs in the SOZ were loosely locked to the slow-wave at 1 Hz but tightly locked to 3 Hz [38]. Similar to the slow-wave in children being shifted to the delta and theta frequency ranges in adults, it may be that the ≧3 Hz locking during non-seizure activity is also shifted into a higher frequency range. The LFO-HFO coupling may be the result of neocortex near-field potentials rather than far-field potentials generated by subcortical structures.

Other studies have investigated the coexistence of LFOs and HFOs in adult populations. One study examined ictal baseline shifts (IBS) in six patients [37]. They found that the onset of HFOs in the high gamma or ripple frequency range either preceded or followed IBS within 300 ms. Moreover, HFOs and IBS were found to have a smaller distribution compared to conventional frequency activity (1 to 70 Hz), a similar finding of another study [52]. This study also found that smaller SOZs and more complete resection of the HFO and IBS contacts correlated with a better post-surgical outcome. One study found that IBS and HFOs were observed in 91% and 81%, respectively, of intracranial seizures from their group of fifteen patients [52]. HFO onset followed IBS onset by 11.5 s and 100% of the earliest IBS onsets and 70% of HFO onset were within the SOZ. One study also investigated ictal broadband EEG activity (0.016 to 600 Hz) in sixteen seizures of one TLE patient. Negative slow shifts were found to coexist with 100 to 300 Hz in the SOZ and these slow shifts preceded the HFOs in all sixteen seizures by 1.6 s and conventional initial EEG changes by 20.4 s [25]. This coexistence of the slow shifts and HFOs was observed only in the SOZ.

Although studies were performed in an adult population, they did not investigate LFO-HFO coupling, but rather coexistence. The studies described above investigating this coupling were performed in a child population. The above example investigated LFO-HFO coupling in an adult population with an emphasis on identification of ROls rather than using CFC as a tool for seizure prediction, as was recently done in other studies [2],[3]. In the other studies [2],[3], the patients used presented with ETLE, which has not been as well studied as TLE, and also presents more difficulties for neurologist when identifying the SOZ. A study of 486 seizures from seventy-two patients found for TLE that 90% of SOZs were correctly localized while for ETLE correct localization was 50% [21]. This is in large part due to localized seizures being more prevalent in TLE compared to ETLE, which is dominated more by generalized seizures. The above example explored CFC occurring during these seizures to provide a potential biomarker for identifying ROIs that will facilitate the localization of the EZ.

Although use of an 8×8 electrode grid is described above to obtain EEG data from patients, those of skill in the art will appreciate that alternative electrode grid arrangements may be employed. EEG data may be processed by the computing device off-line after the EEG data has been acquired and stored to memory or may be processed on-line as the EEG data is being acquired.

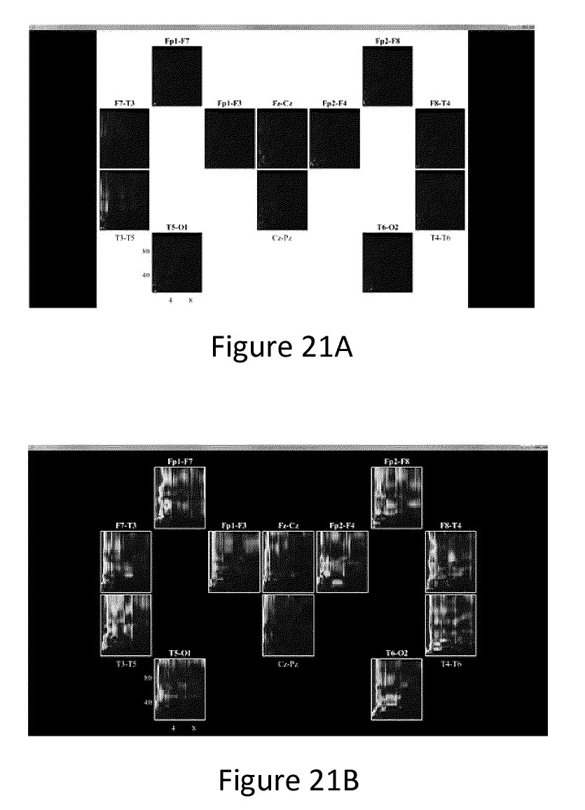

Although in embodiments above the electrodes are described as being cortical electrodes or electrodes implanted on the cortex, those skilled in the art will appreciate that other electrodes may be used. For example, in another embodiment, the electrodes may be scalp electrodes. In this embodiment, method 100 is used to process data received from the scalp electrodes to identify seizure onset and termination times and to identify one or more possible seizure zones of a subject's brain. Exemplary modulation index values calculated based off scalp electrode data recorded during non-seizure and seizure activity are shown in FIGS. 21A and 12B, respectively.

Although embodiments have been described with reference to the accompanying drawings, those of skill in the art will appreciate that variations and modifications may be made without departing from the scope thereof as defined by the appended claims.

### REFERENCES

[1] Akiyama, T, McCoy, B, Go, C Y, Ochi, A, Elliott, I M, Akiyama, M, Donner, E J, Weiss, S K, Snead, O C & Rutka, J T 2011, ‘Focal resection of fast ripples on extraoperative intracranial EEG improves seizure outcome in pediatric epilepsy’, Epilepsia , vol. 52, no. 10, Wiley Online Library, pp. 1802to1811.
[2] Alvarado-Rojas, C, Valderrama, M, Fouad-Ahmed, A, Feldwisch-Drentrup, H, Ihle, M, Teixeira, C A, Sales, F, Schulze-Bonhage, A, Adam, C, Dourado, A, Charpier, S, Navarro, V & Le Van Quyen, M 2014, ‘Slow modulations of high-frequency activity (40to140 Hz) discriminate preictal changes in human focal epilepsy’, Scientific Reports, vol. 4.
[3] Alvarado-Rojas, C, Valderrama, M, Witon, A, Navarro, V & Van Quyen, M L 2011, ‘Probing cortical excitability using cross-frequency coupling in intracranial EEG recordings: A new method for seizure prediction’, Proceedings of the Annual International Conference of the IEEE Engineering in Medicine and Biology Society, EMBS, IEEE, pp. 1632to1635.
[4] Bagshaw, A P, Jacobs, J, LeVan, P, Dubeau, F & Gotman, J 2009, ‘Effect of sleep stage on interictal high-frequency oscillations recorded from depth macroelectrodes in patients with focal epilepsy’, Epilepsia, vol. 50, no. 4, pp. 617to628.
[5] Beleza, P, Bilgin, & Noachtar, S 2009, ‘Interictal rhythmical midline theta differentiates frontal from temporal lobe epilepsies’, Epilepsia, vol. 50, no. 3, pp. 550to555.
[6] Benjamini, Y & Hochberg, Y 1995, ‘Controlling the false discovery rate: a practical and powerful approach to multiple testing’, Journal of the Royal Statistical Society. Series B (Methodological), JSTOR, pp. 289to300.
[7] Bonfiglio, L, Olcese, U, Rossi, B, Frisoli, A, Arrighi, P, Greco, G, Carozzo, S, Andre, P, Bergamasco, M & Carboncini, M C 2012, ‘Cortical source of blink-related delta oscillations and their correlation with levels of consciousness’, Human Brain Mapping, pp. n/aton/a.
[8] Bragin, A, Wilson, C L & Engel, J 2000, ‘Chronic epileptogenesis requires development of a network of pathologically interconnected neuron clusters: a hypothesis’, Epilepsia, vol. 41, no. s6, Wiley Online Library, pp. S144toS152.
[9] Brigo, F 2011, ‘Intermittent rhythmic delta activity patterns’, YEBEH, vol. 20, no. 2, Elsevier Inc., pp. 254to256.
[10] Burnos, S, Hilfiker, P, Sürücü, Scholkmann, F, Krayenbühl, N, Grunwald, T & Sarnthein, J 2014, ‘Human Intracranial High Frequency Oscillations (HFOs) Detected by Automatic Time-Frequency Analysis’, S Charpier (ed.), PLoS ONE, vol. 9, no. 4, p. e94381.
[11] Buzstki, G, Horvath, Z, Urioste, R, Hetke, J & Wise, K 1992, ‘High-frequency network oscillation in the hippocampus’, Science, vol. 256, no. 5059, American Association for the Advancement of Science, pp. 1025to 1027 .
[12] Canolty, R T, Edwards, E, Dalal, S S, Soltani, M, Nagarajan, S S, Kirsch, H E, Berger, M S, Barbaro, N M & Knight, R T 2006, ‘High Gamma Power Is Phase-Locked to Theta Oscillations in Human Neocortex’, Science, vol. 313, no. 5793, pp. 1626 to1628.
[13] Chen, A C N, Feng, W, Zhao, H, Yin, Y & Wang, P 2008, ‘EEG default mode network in the human brain: Spectral regional field powers’, Neurolmage, vol. 41, no. 2, pp. 561to574.
[14] Clemens, B, Bessenyei, M, Fekete, I, Pusk‡s, S, Kond‡kor, I, T-th, M & Holl-dy, K 2010, ‘Theta EEG source localization using LORETA in partial epilepsy patients with and without medication’, Clinical Neurophysiology, vol. 121, no. 6, International Federation of Clinical Neurophysiology, pp. 848to858.
[15] Clemens, Z, Molle, M, Eross, L, Barsi, P, Halasz, P & Born, J 2007, ‘Temporal coupling of parahippocampal ripples, sleep spindles and slow oscillations in humans’, Brain, vol. 130, no. 11, pp. 2868to2878.
[16] Diehl, B & Lüders, HO 2000, ‘Temporal lobe epilepsy: when are invasive recordings needed?’, Epilepsia, vol. 41, no. s3, Wiley Online Library, pp. S61to74.
[17] Dvorak, D & Fenton, A A 2014, ‘Toward a proper estimation of phasetoamplitude coupling in neural oscillations’, Journal ofNeuroscience Methods, vol. 225, Elsevier B. V., pp. 42to56.
[18] Engel, J, Jr, Bragin, A, Staba, R J & Mody, I 2009, ‘High-frequency oscillations: What is normal and what is not?’, Epilepsia, vol. 50, no. 4, pp. 598to604.
[19] Engel, J J 1993, ‘Update on surgical treatment of the epilepsies. Summary of the Second International Palm Desert Conference on the Surgical Treatment of the Epilepsies (1992).’, Neurology, vol. 43, no. 8, pp. 1612to1617.
[20] Foffani, G, Uzcategui, Y G, Gal, B & Menendez de la Pride, L 2007, ‘Reduced Spike-Timing Reliability Correlates with the Emergence of Fast Ripples in the Rat Epileptic Hippocampus’, Neuron, vol. 55, no. 6, pp. 930to941.
[21] Foldvary, N, Klem, G, Hammel, J, Bingaman, W, Najm, I & Lüders, H 2001, ‘The localizing value of ictal EEG in focal epilepsy’, Neurology, vol. 57, no. 11, AAN Enterprises, pp. 2022to2028.
[22] Gambardella, A, Gotman, J, Cendes, F & Andermann, F 1995, ‘Focal intermittent delta activity in patients with mesiotemporal atrophy: a reliable marker of the epileptogenic focus’, Epilepsia, vol. 36, no. 2, Wiley Online Library, pp. 122to129.
[23] Hughes, J R & Fino, J J 2004, ‘EEG in seizure prognosis: association of slow wave activity and other factors in patients with apparent misleading epileptiform findings.’, Clinical EEG and neuroscience: official journal of the EEG and Clinical Neuroscience Society (ENCS), vol. 35, no. 4, pp. 181to184.
[24] Ibrahim, G M, Wong, S M, Anderson, R A, Singh-Cadieux, G, Akiyama, T, Ochi, A, Otsubo, H, Okanishi, T, Valiante, T A, Donner, E, Rutka, J T, Snead, O C, III & Doesburg, SM 2014, ‘Dynamic modulation of epileptic high frequency oscillations by the phase of slower cortical rhythms’, Experimental Neurology, vol. 251, no. C, Elsevier Inc., pp. 30to38.
[25] Imamura, H, Matsumoto, R, Inouchi, M, Matsuhashi, M, Mikuni, N, Takahashi, R & Ikeda, A 2011, ‘Ictal wideband ECoG: Direct comparison between ictal slow shifts and high frequency oscillations’, Clinical Neurophysiology, vol. 122, no. 8, International Federation of Clinical Neurophysiology, pp. 1500 to1504.
[26] Jacobs, J, LeVan, P, Chander, R, Hall, J, Dubeau, F & Gotman, J 2008, ‘Interictal high-frequency oscillations (80-5 00 Hz) are an indicator of seizure onset areas independent of spikes in the human epileptic brain’, Epilepsia, vol. 49, no. 11, pp. 1893to1907.
[27] Jacobs, J, Staba, R, Asano, E, Otsubo, H, Wu, J Y, Zijlmans, M, Mohamed, I, Kahane, P, Dubeau, F, Navarro, V & Gotman, J 2012, ‘High-frequency oscillations (HFOs) in clinical epilepsy’, Progress in Neurobiology, vol. 98, no. 3, Elsevier Ltd, pp. 302to315.
[28] Jacobs, J, Zijlmans, M, Zelmann, R, Châtillon, C-E, Hall, J, Olivier, A, Dubeau, F & Gotman, J 2010, ‘High-frequency electroencephalographic oscillations correlate with outcome of epilepsy surgery’, Annals of Neurology, vol. 67, no. 2, pp. 209to220.
[29] Jefferys, J G R, Menendez de la Prida, L, Wendling, F, Bragin, A, Avoli, M, Timofeev, I & da Silva, F H L 2012, ‘Mechanisms of physiological and epileptic HFO generation’, Progress in Neurobiology, vol. 98, no. 3, Elsevier Ltd, pp. 250to264.
[30] Jones, M S & Barth, DS 1999, ‘Spatiotemporal organization of fast (>200 Hz) electrical oscillations in rat vibrissa/barrel cortex’, Journal of Neurophysiology, vol. 82, no. 3, [Bethesda, Md., etc.] American Physiological Society [etc.], pp. 1599to1609.
[31] Jones, M S, MacDonald, K D, Choi, B, Dudek, F E & Barth, D S 2000, ‘Intracellular correlates of fast (>200 Hz) electrical oscillations in rat somatosensory cortex’, Journal ofNeurophysiology, vol. 84, no. 3, Am Physiological Soc, pp. 1505to1518.
[32] Kandel, A & Buzsáki, G 1997, ‘Cellulartosynaptic generation of sleep spindles, spike-and-wave discharges, and evoked thalamocortical responses in the neocortex of the rat’, The Journal ofNeuroscience, vol. 17, no. 17, Soc Neuroscience, pp. 6783to6797.
[33] Kerber, K, Dümpelmann, M, Schelter, B, Le Van, P, Korinthenberg, R, Schulze-Bonhage, A & Jacobs, J 2013, ‘Differentiation of specific ripple patterns helps to identify epileptogenic areas for surgical procedures’, Clinical Neurophysiology, International Federation of Clinical Neurophysiology, pp. 1to7.
[34] Khamis, H, Mohamed, A & Simpson, S 2009, ‘Seizure state detection of temporal lobe seizures by autoregressive spectral analysis of scalp EEG’, Clinical Neurophysiology, vol. 120, no. 8, International Federation of Clinical Neurophysiology, pp. 1479to1488.
[35] Lévesque, M, Bortel, A, Gotman, J & Avoli, M 2011, ‘High-frequency (80to500Hz) oscillations and epileptogenesis in temporal lobe epilepsy’, Neurobiology ofDisease, vol. 42, no. 3, Elsevier Inc., pp. 231to241.
[36] Melani, F, Zelmann, R, Mari, F & Gotman, J 2013, ‘Continuous High Frequency Activity: A peculiar SEEG pattern related to specific brain regions’, Clinical Neurophysiology, vol. 124, no. 8, International Federation of Clinical Neurophysiology, pp. 1507to1516.
[37] Modur, P N, Vitaz, T W & Zhang, S 2012, ‘Seizure Localization Using Broadband EEG’, Journal of clinical neurophysiology, vol. 29, no. 4, pp. 309to319.
[38] Nariai, H, Matsuzaki, N, Juhász, C, Nagasawa, T, Sood, S, Chugani, H T & Asano, E 2011, ‘Ictal high -frequency oscillations at 80-200Hz coupled with delta phase in epileptic spasms’, Epilepsia, vol. 52, no. 10, pp. e130to e 1 3 4 .
[39] Nolte, G, Bai, O, Wheaton, L, Mari, Z, Vorbach, S & Hallett, M 2004, ‘Identifying true brain interaction from EEG data using the imaginary part of coherency’, Clinical Neurophysiology, vol. 115, no. 10, pp. 2292to2307.
[40] Otsubo, H, Ochi, A, Imai, K, Akiyama, T, Fujimoto, A, Go, C, Dirks, P & Donner, E J 2008, ‘High-frequency oscillations of ictal muscle activity and epileptogenic discharges on intracranial EEG in a temporal lobe epilepsy patient’, Clinical Neurophysiology, vol. 119, no. 4, pp. 862to868.
[41] Panet-Raymond, D & Gotman, J 1990, ‘Asymmetry in delta activity in patients with focal epilepsy’, Electroencephalography and clinical neurophysiology, vol. 75, no. 6, Elsevier, pp. 474to481.
[42] Rosenow, F & Lüders, H 2001, ‘Presurgical evaluation of epilepsy’, Brain, vol. 124, no. 9, Oxford Univ Press, pp. 1683to1700.
[43] Schnitzler, A & Gross, J 2005, ‘Normal and pathological oscillatory communication in the brain’, Nature Reviews Neuroscience, vol. 6, no. 4, pp. 285to296.
[44] Spencer, S & Huh, L 2008, ‘Outcomes of epilepsy surgery in adults and children’, The Lancet Neurology, vol. 7, no. 6, Elsevier, pp. 525to537.
[45] Tao, J X, Chen, X-J, Baldwin, M, Yung, I, Rose, S, Frim, D, Hawes-Ebersole, S & Ebersole, J S 2011, ‘Interictal regional delta slowing is an EEG marker of epileptic network in temporal lobe epilepsy’, Epilepsia, vol. 52, no. 3, pp. 467to476.
[46] Theiler, J, Eubank, S, Longtin, A, Galdrikian, B & Doyne Farmer, J 1992, ‘Testing for nonlinearity in time series: the method of surrogate data’, Physica D: Nonlinear Phenomena, vol. 58, no. 1, Elsevier, pp. 77to94.
[47] Tort, A B L, Komorowski, R, Eichenbaum, H & Kopell, N J 2010, ‘Measuring Phase-Amplitude Coupling Between Neuronal Oscillations of Different Frequencies’, Journal of Neurophysiology, vol. 104, no. 2, pp. 1195to1210.
[48] Tort, A B L, Scheffer-Teixeira, R, Souza, B C, Draguhn, A & Brankaek, J 2013, ‘Theta-associated high-frequency oscillations (110to160Hz) in the hippocampus and neocortex’, Progress in Neurobiology, vol. 100, Elsevier Ltd, pp. 1to14.
[49] Urrestarazu, E, Chander, R, Dubeau, F & Gotman, J 2007, ‘Interictal high-frequency oscillations (100 500 Hz) in the intracerebral EEG of epileptic patients’, Brain, vol. 130, no. 9, pp. 2354to2366.
[50] Vanrumste, B, Jones, R D, Bones, P J & Carroll, G J 2005, ‘Slow-wave activity arising from the same area as epileptiform activity in the EEG of paediatric patients with focal epilepsy’, Clinical Neurophysiology, vol. 116, no. 1, pp. 9to17.
[51] Wu, J Y, Sankar, R, Lerner, J T, Matsumoto, J H, Vinters, H V & Mathern, G W 2010, ‘Removing interictal fast ripples on electrocorticography linked with seizure freedom in children’, Neurology, vol. 75, no. 19, AAN Enterprises, pp. 1686to1694.
[52] Wu, S, Kunhi Veedu, H P, Lhatoo, S D, Koubeissi, M Z, Miller, J P & L Ÿders, H O 2014, ‘Role of ictal baseline shifts and ictal high-frequency oscillations in stereo-electroencephalography analysis of mesial temporal lobe seizures’, Epilepsia, vol. 55, no. 5, pp. 690to698.
[53] Ylinen, A, Bragin, A, N‡dasdy, Z, Jand-, G, Szabo, I, Sik, A & Buzs‡ki, G 1995, ‘Sharp wave-associated high-frequency oscillation (200 Hz) in the intact hippocampus: network and intracellular mechanisms’, The Journal of Neuroscience, vol. 15, no. 1, Soc Neuroscience, pp. 30to46.
[54] Hjorth, B 1975, ‘An on-line transformation of EEG scalp potentials into orthogonal source derivations’, Electroencephalography and clinical neurophysiology, vol. 39, no. 5, Elsevier, pp. 526to530.
[55] Huang, N E, Shen, Z, Long, S R, Wu, M, Shih, H, Zheng, Q, Yen, N-C, Tung, C C & Liu, H 1998, ‘The Empirical Mode Decomposition and the Hilbert Spectrum for Nonlinear and Non-Stationary Time Series Analysis’, Proceedings of the Royal Society A: Mathematical, Physical and Engineering Sciences, vol. 454, pp. 903to995.
[56] Wu, Z & Huang, N E 2009, ‘Ensemble empirical mode decomposition: A noise-assisted data analysis method’, Advances in Adaptive Data Analysis, vol. 1, no. 01, World Scientific, pp. 1to41.

1-32. (canceled)

33. A computer-implemented method of processing electroencephalogram (EEG) signals received from a plurality of electrodes, wherein the received EEG signals are from flames relative to clinical seizure onset, mid-seizure, and seizure termination timestamps, the method comprising:
pre-processing the received EEG signals to produce a plurality of channels, wherein a channel corresponds to the difference between an electrode and a reference;
processing the channels to determine a modulation index value for each channel;
identifying one or more channels that have a modulation index value above a threshold level observed during each frame; and
identifying one or more possible regions of interest corresponding to seizure zones of a subject's brain using the identified one or more channels that have a modulation index value above the threshold level.

34. The method of claim 33, wherein the reference is an adjacent electrode.

35. The method of claim 33, where processing the channels to determine a modulation index value comprises:
modulating amplitudes of high-frequency oscillations of the EEG signals by phases of low-frequency oscillations of the EEG signals.

36. The method of claim 35 wherein the high-frequency oscillations comprise frequencies between 11 Hz and 450 Hz.

37. The method of claim 35 wherein the low-frequency oscillations comprise frequencies between 0.5 Hz and 10 Hz.

38. The method of claim 33 further comprising:
calculating cross-channel modulation index valves.

39. The method of claim 38 further comprising:
determining one or more channels pairs that have eigenvalue decomposed cross-channel modulation index value above another threshold level for a period of time.

40. The method of claim 38 further comprising:
eigenvalue decomposing the calculated cross-channel modulation index values to determine a number of eigenvalues and associated eigenvectors;
calculating a mean of the components in a selected eigenvector; and
setting the threshold level as three standard errors of mean above the calculated mean of the components in the selected eigenvector.

41. The method of claim 33 wherein the threshold level is approximately 0.3 times a maximum modulation index value.

42. The method of claim 33, wherein the one or more possible regions of interest correspond to one or more areas for surgical resection.

43. The method of claim 42, further comprising surgically resecting at least a portion of the region of interest.

44. The method of claim 33, further comprising implanting the plurality of electrodes in a cortex of the subject's brain.

45. An apparatus comprising:
memory storing executable instructions; and
at least one processor communicating with the memory and executing the instructions therein to cause the apparatus at least to:
receive electroencephalogram (EEG) signals from a plurality of electrodes, wherein the received EEG signals arc from frames relative to clinical seizure onset, mid-seizure, and seizure termination timestamps;
pre-process the received EEG signals to produce a plurality of channels, wherein a channel corresponds to the difference between an electrode and a reference;
process the BEG channels, to determine a modulation index value for each channel;
identify one or more channels that have a modulation index value above a threshold level observed during each frame; and
identify one or more possible regions of interest corresponding to seizure zones of a subject's brain using the identified one or more channels that have a modulation index value above the threshold level.

46. A method of identifying one or more possible surgical resection sites corresponding to seizure zones in a subject's brain for treating seizures in a subject, the method comprising:
pre-processing electroencephalogram (EEG) signals received from a plurality electrodes, wherein the received EEG signals are from frames relative to clinical seizure onset, mid-seizure, and seizure termination timestamps, to produce a plurality of channels, wherein a channel corresponds to the difference between an electrode and a reference;
processing the channels to determine a modulation index value for each electrode;
identifying one or more channels that have a modulation index value above a threshold level observed during each frame; and
identifying the one or more possible surgical resection sites using the identified one or more channels that have a modulation index value above the threshold level.

47. The method of claim 46, wherein the reference is an adjacent electrode.

48. The method of claim 46, where processing the channels to determine a modulation index value comprises:
modulating amplitudes of high-frequency oscillations of the EEG signals by phases of low-frequency oscillations of the EEG signals.

49. The method of claim 48 wherein the high-frequency oscillations comprise frequencies between 11 Hz and 450 Hz.

50. The method of claim 48 wherein the low-frequency oscillations comprise frequencies between 0.5 Hz and 10 Hz.

51. The method of claim 46 thither comprising:
calculating cross-channel modulation index values.

52. The method of claim 51 further comprising:
determining one or more channels pairs that have eigenvalue decomposed cross-channel modulation index value above another threshold level for a period of time.

53. The method of claim 51 further comprising:
eigenvalue decomposing the calculated cross-channel modulation index values to determine a number of eigenvalues and associated eigenvectors;
calculating a mean of the components in a selected eigenvector; and
setting the threshold level as three standard errors of mean above the calculated mean of the components in the selected eigenvector.

54. The method of claim 46 wherein the threshold level is approximately 0.3 times a maximum modulation index value.

55. The method of claim 46, wherein the one or more possible regions of interest correspond to one or more areas for surgical resection.

56. The method of claim 55, further comprising surgically resecting at least a portion of the region of interest.

57. The method of claim 56, further comprising implanting the plurality of electrodes in a cortex of the subject's brain.
